(19) United States
(12) Patent Application Publication
(10) Pub. No.: US 2025/0090011 A1
(43) Pub. Date: Mar. 20, 2025

(54) MEDICAL INSTRUMENT WITH TRANSLATING ACTUATOR TO STEER SHAFT

(71) Applicant: Acclarent, Inc., Irvine, CA (US)

(72) Inventors: Raymond Yue-sing TANG, Rosemead, CA (US); Shubhayu BASU, Anaheim, CA (US); Henry F. SALAZAR, Pico Rivera, CA (US); Hao H. TRAN, Santa Ana, CA (US); Zachary RADNER, Anaheim, CA (US); Cesar FUENTES-ORTEGA, Pasadena, CA (US)

(21) Appl. No.: 18/890,750

(22) Filed: Sep. 19, 2024

Related U.S. Application Data
(60) Provisional application No. 63/539,363, filed on Sep. 20, 2023.

(51) Int. Cl. A61B 1/233; A61B 1/005; A61B 1/05; A61B 1/06; A61B 5/06; A61M 29/02

(52) U.S. Cl. CPC A61B 1/233; A61B 1/0052; A61B 1/0055; A61B 1/0057; A61B 1/05; A61B 1/0676; A61B 5/065; A61M 29/02

(57) ABSTRACT

Catheter shafts (such as guide catheter or dilation catheter shafts) disclosed herein can include a rigid proximal portion, a deflectable distal portion located distally from the proximal portion, and a pull wire extending from the deflectable distal portion along the proximal portion. The pull wire can drive the deflectable distal portion between a straight configuration and a bent configuration, which makes the catheter system adaptable for use with both pediatric and adult patients. Specifically, because the pull wire facilitates changing a bend angle of the deflectable distal portion of the catheter shaft, the catheter can fit in a wide variety of differently sized nasal cavities and Eustachian Tubes. Optionally, a visualizing distal tip can be positioned distally from the deflectable distal portion of the catheter shaft and include a distally facing camera that can be used to confirm that the deflectable distal portion is properly positioned in the patient.

### CROSS-REFERENCE TO RELATED APPLICATIONS

This application claims the benefit of U.S. Provisional Application No. 63/539,363, filed Sep. 20, 2023, the entirety of which is incorporated herein by reference.

### BACKGROUND

Referring to FIGS. 1A-1B, the ear 10 is divided into three parts: an external ear 12, a middle ear 14 and an inner ear 16. The external ear 12 consists of an auricle 18 and ear canal 20 that gather sound and direct it toward a tympanic membrane 22 (also referred to as the eardrum) located at an inner end 24 of the ear canal 20. The middle ear 14 lies between the external and inner ears 12, 16 and is connected to the back of the throat by a Eustachian tube (ET) 26, which serves as a pressure equalizing valve between the ear 10 and the sinuses. The ET 26 terminates in a pharyngeal ostium 28 in the nasopharynx region 30 of the throat 32. In addition to the eardrum 22, the middle ear 14 also consists of three small ear bones (ossicles): the malleus 34 (hammer), incus 36 (anvil) and stapes 38 (stirrup). These bones 34, 36, 38 transmit sound vibrations to the inner ear 16 and thereby act as a transformer, converting sound vibrations in the canal 20 of the external ear 12 into fluid waves in the inner ear 16. These fluid waves stimulate several nerve endings 40 that, in turn, transmit sound energy to the brain where it is interpreted.

The ET 26 is a narrow, one-and-a-half inch long channel connecting the middle ear 14 with the nasopharynx 30, the upper throat area just above the palate, in back of the nose. The ET 26 functions as a pressure equalizing valve for the middle ear 14, which is normally filled with air. When functioning properly, the ET 26 opens for a fraction of a second periodically (about once every three minutes) in response to swallowing or yawning. In so doing, it allows air into the middle ear 14 to replace air that has been absorbed by the middle ear lining (mucous membrane) or to equalize pressure changes occurring on altitude changes. Anything that interferes with this periodic opening and closing of the ET 26 may result in hearing impairment or other ear symptoms.

Obstruction or blockage of the ET 26 results in a negative middle ear 14 pressure, with retraction (sucking in) of the eardrum 22. In adults, this is usually accompanied by some ear discomfort, a fullness or pressure feeling and may result in a mild hearing impairment and head noise (tinnitus). There may be no symptoms in children. If the obstruction is prolonged, fluid may be drawn from the mucous membrane of the middle ear 14, creating a condition referred to as serous otitis media (fluid in the middle ear). This may occur frequently in children in connection with an upper respiratory infection and account for hearing impairment associated with this condition.

When the ET 26 is blocked, the body may absorb the air from the middle ear 14, causing a vacuum to form that tends to pull the lining membrane and ear drum 22 inwardly, causing pain. Next, the body may replace the vacuum with more fluid which tends to relieve the pain, but the patient can experience a fullness sensation in the ear 10. Finally, the fluid can become infected, which can lead to pain, illness, and temporary hearing loss. If the inner ear 14 is affected, the patient may feel a spinning or turning sensation (vertigo).

Methods for treating the middle ear 14 and restriction or blockage of the ET 26 include those disclosed in U.S. Pat. No. 10,206,821, entitled “Eustachian Tube Dilation Balloon with Ventilation Path,” issued Feb. 19, 2019; U.S. Pat. No. 10,350,396, entitled “Vent Cap for a Eustachian Tube Dilation System,” issued Jun. 29, 2019; and U.S. Pat. No. 11,013,896, entitled “Method and System for Eustachian Tube Dilation,” issued May 25, 2021. The disclosures of U.S. Pat. No. 10,206,821, entitled “Eustachian Tube Dilation Balloon with Ventilation Path,” issued Feb. 19, 2019; U.S. Pat. No. 10,350,396, entitled “Vent Cap for a Eustachian Tube Dilation System,” issued Jun. 29, 2019; and U.S. Pat. No. 11,013,896, entitled “Method and System for Eustachian Tube Dilation,” issued May 25, 2021, are incorporated in their entireties herein.

While a variety of instruments have been made and used, it is believed that no one prior to the inventors has made or used the inventions described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1A depicts a cross-sectional view of an adult human ear showing the inner, middle and outer ear portions and the Eustachian tube connecting the middle ear with the nasopharynx region of the throat, with an illustrative guide catheter and balloon dilation catheter positioned in relation to the Eustachian tube of a patient, with the balloon dilation catheter in a deflated configuration, according to some embodiments.

FIG. 1B depicts a cross-sectional view of the adult human ear of FIG. 1A, with the illustrative guide catheter and balloon dilation catheter of FIG. 1A positioned in relation to the Eustachian tube of a patient, with the balloon dilation catheter in an inflated configuration, according to some embodiments.

FIG. 2 depicts a cross-sectional view of a pediatric human ear showing the inner, middle and outer ear portions and the Eustachian tube connecting the middle ear with the nasopharynx region of the throat, according to some embodiments.

FIG. 3 depicts a perspective view of an illustrative guide catheter that may be used to access the Eustachian tube of a pediatric human, according to some embodiments.

FIG. 4 depicts an exploded perspective view of a handle assembly of the guide catheter of FIG. 3, according to some embodiments.

FIG. 5 depicts an enlarged perspective view of a deflectable distal portion and a distal tip of the guide catheter of FIG. 3, according to some embodiments.

FIG. 6A depicts a cross-sectional view, taken along line 6-6 of FIG. 3, of the guide catheter of FIG. 3, with the deflectable distal portion of FIG. 5 in a straight, non-articulated, configuration, according to some embodiments.

FIG. 6B depicts a cross-sectional view, taken along line 6-6 of FIG. 3, of the guide catheter of FIG. 3, with the deflectable distal portion of FIG. 5 in a bent, articulated, configuration, according to some embodiments.

FIG. 7 depicts a cross-sectional view of an alternative deflectable guide catheter having an alternative handle assembly, according to some embodiments.

FIG. 8 depicts a cross-sectional view of another alternative deflectable guide catheter, according to some embodiments.

FIG. 9 depicts a perspective view of an illustrative dilation catheter, according to some embodiments.

FIG. 10A depicts an elevational side view of the distal end of the dilation catheter of FIG. 9, where the distal end is in a straight, non-articulated, configuration, according to some embodiments.

FIG. 10B depicts an elevational side view of the distal end of the dilation catheter of FIG. 9, where the distal end is in a bent, articulated, configuration, according to some embodiments.

FIG. 11 depicts a cross-sectional view of the dilation catheter of FIG. 9, taken along line 11-11 of FIG. 9, where a camera and a plurality of light sources are disposed within a working channel of an elongate shaft of the dilation catheter, according to some embodiments.

FIG. 12 depicts a cross-sectional view of the dilation catheter of FIG. 9, taken along line 11-11 of FIG. 9, where a camera, a plurality of light sources, and a position sensor are disposed within the working channel of the elongate shaft of the dilation catheter, according to some embodiments.

FIG. 13 depicts a cross-sectional view of the dilation catheter of FIG. 9, taken along line 11-11 of FIG. 9, where a guidewire is disposed within an elongate shaft of the dilation catheter, according to some embodiments.

FIG. 14A depicts a cross-sectional side view of the guide catheter of FIG. 3 and the dilation catheter of FIG. 9 loaded within the guide catheter, where the guide catheter and the dilation catheter are located within the nasopharynx region of the throat illustrated in FIG. 2, where the deflectable distal portion of the guide catheter and the distal end of the dilation catheter each in the straight, non-articulated, configuration, according to some embodiments.

FIG. 14B depicts a cross-sectional side view of the guide catheter of FIG. 3 and the dilation catheter of FIG. 9 loaded within the guide catheter, where the guide catheter and the dilation catheter are located within the nasopharynx region of the throat illustrated in FIG. 2, with the deflectable distal portion of the guide catheter and the distal end of the dilation catheter each in the bent, articulated, configuration, according to some embodiments.

FIG. 14C depicts a cross-sectional side view of the guide catheter of FIG. 3 located within the nasopharynx region of the throat illustrated in FIG. 2, where the dilation catheter of FIG. 9 is advanced into the Eustachian tube of FIG. 2 in an un-dilated configuration, with the deflectable distal portion of the guide catheter and the distal end of the dilation catheter each in the bent, articulated, configuration, according to some embodiments.

FIG. 14D depicts a cross-sectional side view of the guide catheter of FIG. 3 located within the nasopharynx region of the throat illustrated in FIG. 2, where the dilation catheter of FIG. 9 is located within the Eustachian tube of FIG. 2 in a dilated configuration, with the deflectable distal portion of the guide catheter and the distal end of the dilation catheter each in the bent, articulated, configuration, according to some embodiments.

FIG. 14E depicts a cross-sectional side view of the guide catheter of FIG. 3 located within the nasopharynx region of the throat illustrated in FIG. 2, where the dilation catheter of FIG. 9 is located within the Eustachian tube of FIG. 2 and returned to the un-dilated configuration, with the deflectable distal portion of the guide catheter and the distal end of the dilation catheter each in the bent, articulated, configuration, according to some embodiments.

FIG. 14F depicts a cross-sectional side view of the guide catheter of FIG. 3 located within the nasopharynx region of the throat illustrated in FIG. 2, where the dilation catheter of FIG. 9 is retracted within the guide catheter in the un-dilated configuration, with the deflectable distal portion of the guide catheter and the distal end of the dilation catheter each in the bent, articulated, configuration, according to some embodiments.

FIG. 14G depicts a cross-sectional side view of the guide catheter of FIG. 3 and the dilation catheter of FIG. 9 located within the nasopharynx region of the throat illustrated in FIG. 2, with the deflectable distal portion of the guide catheter and the distal end of the dilation catheter each returned to the straight, un-articulated, configuration, according to some embodiments.

FIG. 14H depicts a cross-sectional side view of the nasopharynx region of the throat and Eustachian tube illustrated in FIG. 2, with the guide catheter of FIG. 3 and the dilation catheter of FIG. 9 retracted, thereby leaving a dilated Eustachian tube, according to some embodiments.

FIG. 15 depicts a top plan view of an illustrative dilation catheter, according to some embodiments.

FIG. 16 depicts an enlarged top plan view of a distal end of a balloon of the dilation catheter of FIG. 15, according to some embodiments.

FIG. 17 depicts a perspective view of an illustrative dilation catheter, according to some embodiments.

FIG. 18 depicts a perspective view of the distal end of the dilation catheter of FIG. 17, according to some embodiments.

FIG. 19 depicts a perspective view a deflection guide of the shaft assembly of the dilation catheter of FIG. 17, according to some embodiments.

The drawings are not intended to be limiting in any way, and it is contemplated that various embodiments of the inventions may be carried out in a variety of other ways, including those not necessarily depicted in the drawings. The accompanying drawings incorporated in and forming a part of the specification illustrate several aspects of the present inventions, and together with the description serve to explain the principles of the inventions; it being understood, however, that these inventions are not limited to the precise arrangements shown.

### DETAILED DESCRIPTION

The following detailed description should be read with reference to the drawings, in which like elements in different drawings are identically numbered. The drawings, which are not necessarily to scale, depict illustrative examples for the purpose of explanation only and are not intended to limit the scope of the inventions. The detailed description illustrates by way of example, not by way of limitation, the principles of the inventions. This description will clearly enable one skilled in the art to make and use the inventions, and describes several examples, adaptations, variations, alternative and uses of the inventions, including what is presently believed to be the best mode of carrying out the inventions.

As used herein, the terms “about” and “approximately” for any numerical values or ranges indicate a suitable dimensional tolerance that allows the part or collection of components to function for its intended purpose as described herein.

### I. Illustrative Eustachian Tube Catheter System

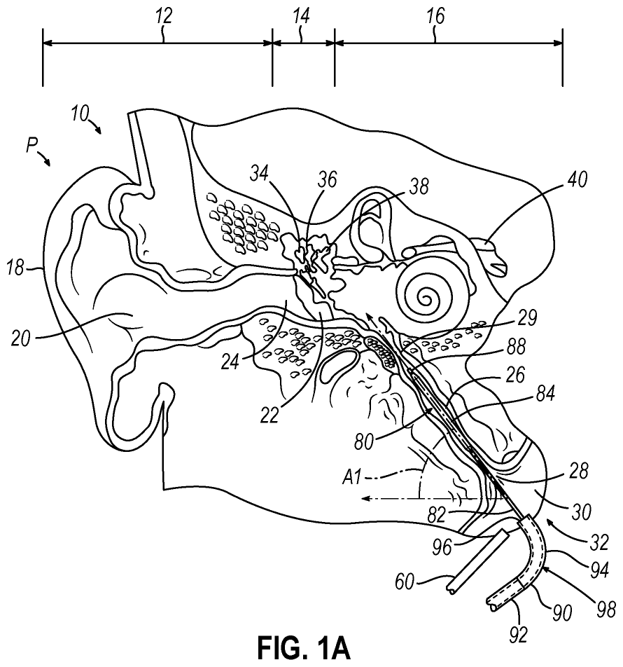

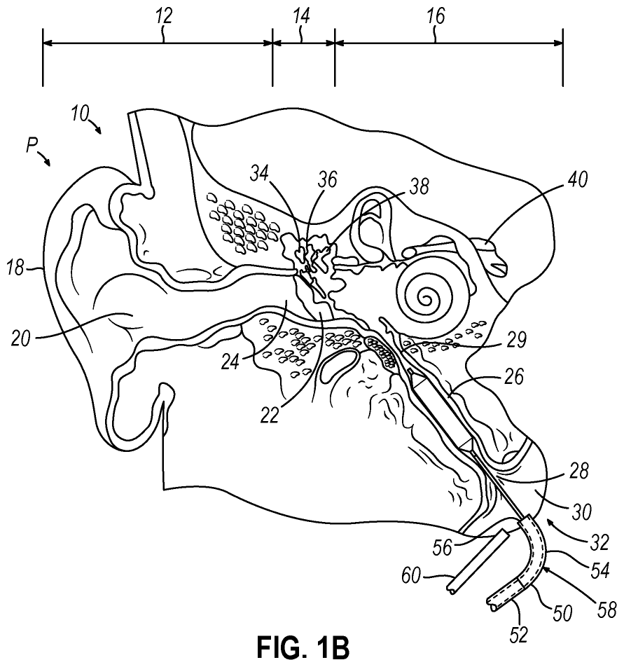

One example of a treatment that may be performed to treat an obstructed and/or blocked ET 26 includes accessing the ET 26 using a guide catheter 90 and a balloon dilation catheter 80, as shown in FIGS. 1A-1B. The guide catheter 90 of the present example includes an elongate tubular shaft 92 that has a proximal end (no shown), a distal end 96, and a lumen 98 therebetween. The guide catheter 90 may have any suitable length, diameter, angle of bend, and location of the bend along the length of the catheter 90, to facilitate accessing an ET 26 opening, such as the pharyngeal ostium 28.

The distal portion of the guide catheter 90 has a preformed, rigid bend 94 with an angle between about 45 degrees and about 65 degrees, and more preferably between about 50 degrees and about 60 degrees, and particularly about 55 degrees, to facilitate access into the ET 26 via the pharyngeal ostium 28. The distal portion of the guide catheter 90, including the bend 94, is made of a transparent material such as a polymer including but not limited to nylon and PTFE such that the balloon dilation catheter 80 is visible within the distal portion and such that distal portion is more flexible than the elongate shaft 92. The distal tip 96 of the guide catheter 90 is made of PEBAX® (polyether block amide) such that it provides for atraumatic access to the ET 26 and may contain 20% barium sulfate or other similar radiopaque materials for visualizable access.

The balloon dilation catheter 80 of the present example generally includes an elongate shaft 82 having a proximal end (not shown) and a distal end 88. The balloon dilation catheter 80 further includes a balloon 84 on the distal end 88 of the elongate shaft 82. The balloon 84 may be a polymer balloon (compliant, semi-compliant, or non-compliant). In some versions, the balloon 84 comprises a suitable non-compliant material such as but not limited to polyethylene terepthalate (PET), PEBAX® (polyether block amide), nylon, or the like. The balloon catheter 80 may include any size of balloon including, but not limited to, balloons of 2 mm to 8 mm in diameter or of between about 5 mm and 6 mm (when inflated) and 12 mm to 24 mm in working length (for example 2 mm×12 mm, 3.5 mm×12 mm, 5 mm×16 mm, 5 mm×24 mm, 6 mm×16 mm, 6 mm×20 mm, 6 mm×24 mm, 7 mm×16 mm and 7 mm×24 mm).

The balloon 84 may be expanded to treat the ET 26 after the balloon 84 is placed in a desirable location in the ET 26, as shown in FIGS. 1A-1B and described in greater detail below. For example, the opening area of the ET 26 includes a pharyngeal ostium 28, and dilation catheter 80 may be advanced to position the balloon 84 within the ET 26 via the pharyngeal ostium 28. An endoscope 60 may be used to assist in positioning the dilation catheter 80. The endoscope 60 may be advanced through the nasal passage to view the dilation catheter 80.

After the balloon 84 is positioned within the ET 26, the balloon 84 may be inflated to an expanded state to thereby dilate the ET 26, as shown in FIG. 1B. The balloon 84 may be held in location while in an expanded state for an extended period of time (e.g., several seconds or minutes). The balloon catheter 80 may also deliver a substance to the ET 26, such as one or more therapeutic or diagnostic agents. The balloon dilation catheter 80 and the guide catheter 90 may be removed from the patient after the balloon 84 has been deflated/unexpanded, thereby leaving a dilated ET 26. The ET 26 may then resume functioning, normally opening and closing to equalize atmospheric pressure in the middle ear 14 and protect the middle ear 14 from unwanted pressure fluctuations and loud sounds.

II. Illustrative Guide Catheter and Dilation Catheter with Steerable Shaft

As mentioned above, and as shown in FIG. 1A-1B, the guide catheter 90 includes a rigid, preformed the bend 94 designed to facilitate access into the ET 26 via the pharyngeal ostium 28. In particular, the bend 94 forms a suitable angle to access the ET 26 of an adult patient such that the distal end 96 is aligned with the ET 26. The angle A1 formed by the long axis of the ET 26 relative to a transverse plane, as well as the anatomical dimensions of an adult patient's nasal cavity, may allow for the bend 94 to be formed of a rigid or semi-rigid material and inserted transnasally in order to suitably align the distal end 96 to provide access to ET 26 without undesirably damaging adjacent anatomical structures. In other words, the bend 94 of the guide catheter 90 may be present while being inserted transnasally within an adult patient to facilitate access to the ET 26.

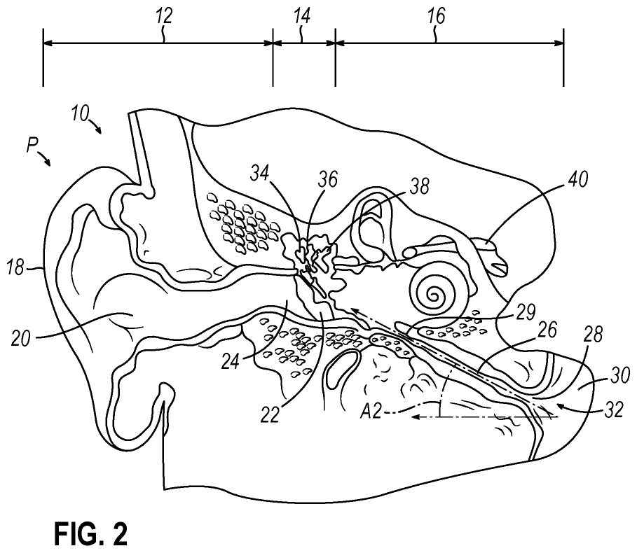

FIG. 2 shows an illustrative ET 26 of a pediatric patient. Pediatric patients may tend to have an ET 26 that extends along a long axis forming a smaller the angle A2 with a transverse plane as compared to the angle A1 (see FIG. 1A) of an adult patient. Due to such a pronounced the angle A2, a bend 94 of the guide catheter 90 that is suitable for an adult patient may not have a sufficient angle to facilitate access of a pediatric patient's ET 26. Therefore, accessing a pediatric patient's ET 26 may warrant a guide catheter with a greater bend angle compared to the bend 94 of the guide catheter 90 described above. However, utilizing a guide catheter 90 with a greater bend angle may not be conducive for inserting the distal end 96 transnasally within a pediatric patient, as this may cause undesirable damage to adjacent anatomical structures during insertion. Further, the nasal cavity of a pediatric patient may be substantially smaller compared to an adult patient, such that a separate the endoscope 60 may not fit within the pediatric nasal cavity simultaneously with the guide catheter 90. Therefore, it may be desirable to provide a guide catheter configured to facilitate access to the ET 26 of a pediatric patient. Further, it may be desirable to provide guide catheter configured to promote visualization within the nasal cavity of the pediatric patient during illustrative use in accordance with the description herein.

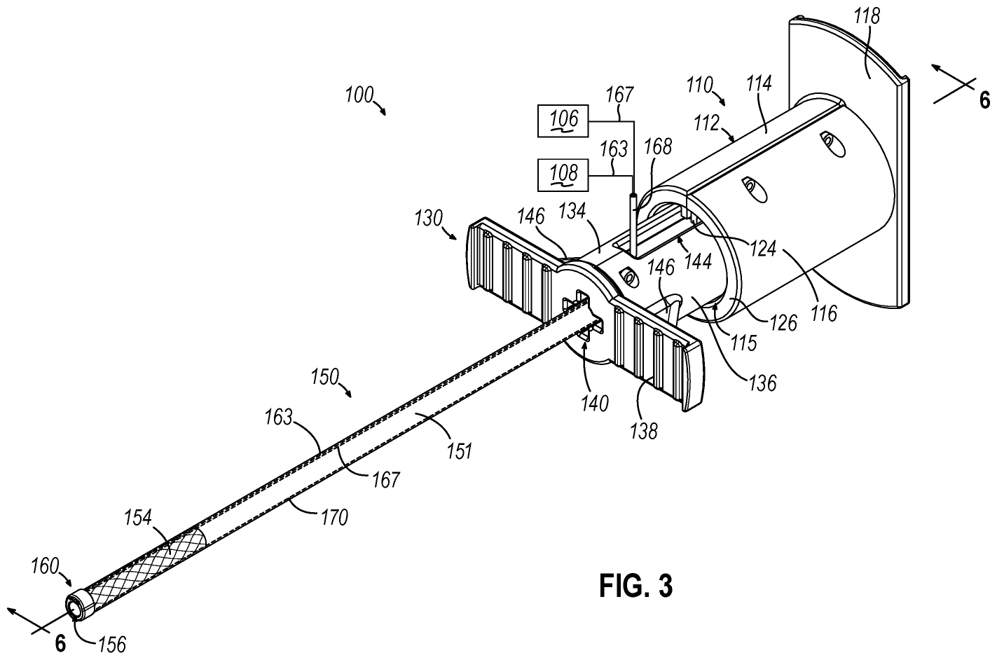

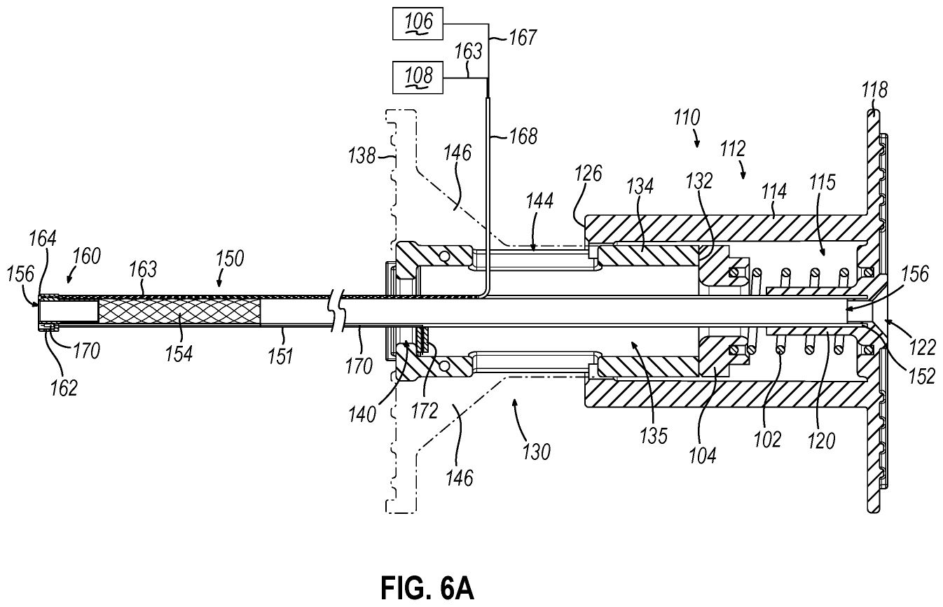

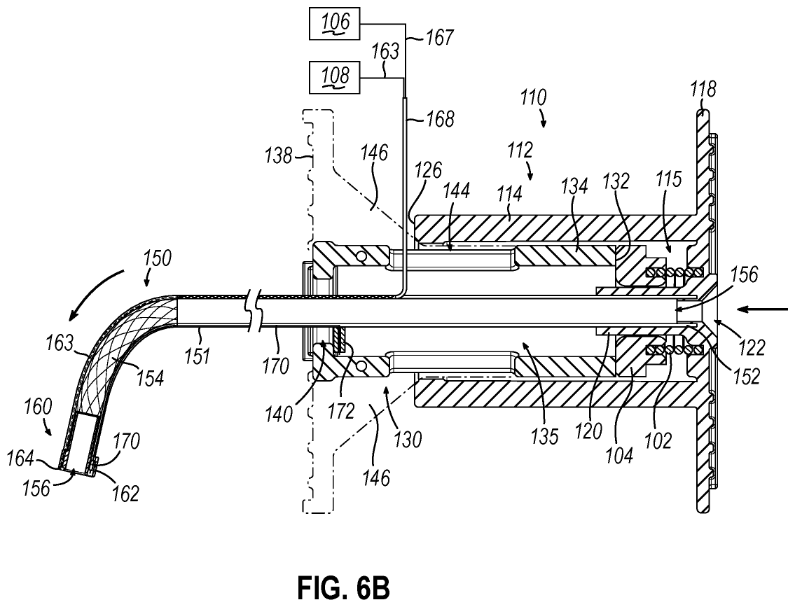

FIG. 3 shows an illustrative guide catheter 100 for use in treating the ET 26 of a pediatric patient. The guide catheter 100 includes a handle assembly 110, an elongate guide catheter shaft 150, and a visualizing distal tip 160. As will be described in greater detail below, a clinician may utilize the handle assembly 110 to selectively deflect a portion of the elongate guide catheter shaft 150 between a substantially straight configuration (see FIGS. 3, 5, and 6A) and a bent configuration (see FIG. 6B) in order to selectively facilitate access to the ET 26 of a pediatric patient, while also providing visualization of the pharyngeal ostium 28.

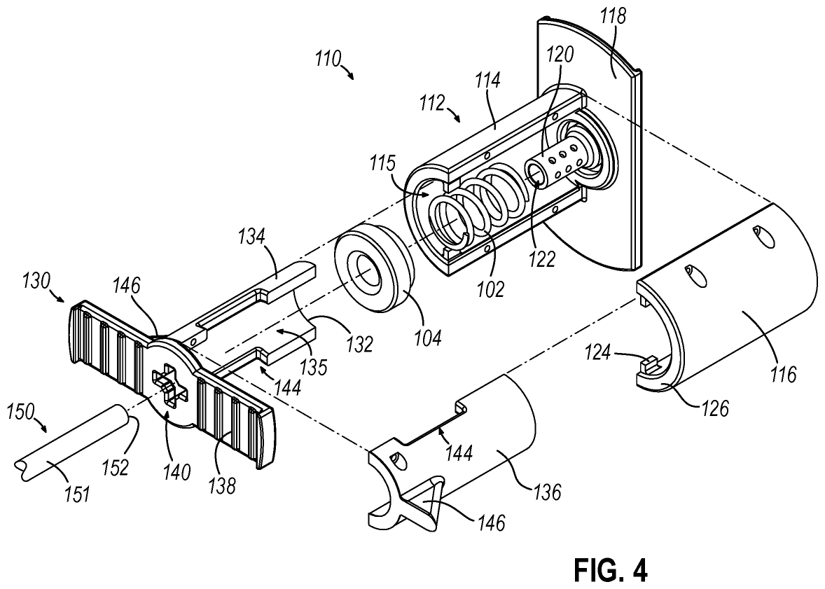

As shown in FIG. 4, the handle assembly 110 includes a proximal handle section 112, a distal handle section 130, a bias spring 102, and a spacer 104. The bias spring 102 and the spacer 104 are housed within an internal chamber 115 defined by the proximal handle section 112. Further, the bias spring 102 and the spacer 104 are interposed between a proximally presented surface 132 of the distal handle section 130 and a proximal flange 118 of the proximal handle section 112 in order to bias the handle sections 112, 130 away from each other. As will be described in greater detail below, a clinician may squeeze the handle sections 112, 130 together with a single hand in order to overcome the bias force of the spring 102 to thereby actuate the handle sections 112, 130 toward each other and controllably deflect the elongate guide catheter shaft 150 between the substantially straight configuration (see FIGS. 3, 5, and 6A) and the bent configuration (see FIG. 6B).

The proximal handle section 112 includes a first body 114 and a second body 116 that mate together in order to define the internal chamber 115. The first body 114 includes a proximal flange 118 having a collar 120 defining a lumen 122 extending within the internal chamber 115. The collar 120 extends within a portion of the spring 102 such that a proximal end of the spring 102 engages a distally presented surface of the flange 118.

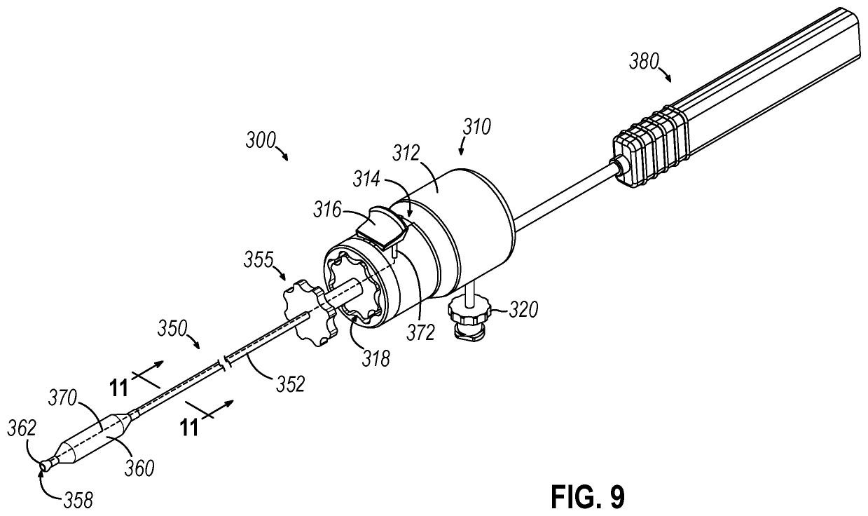

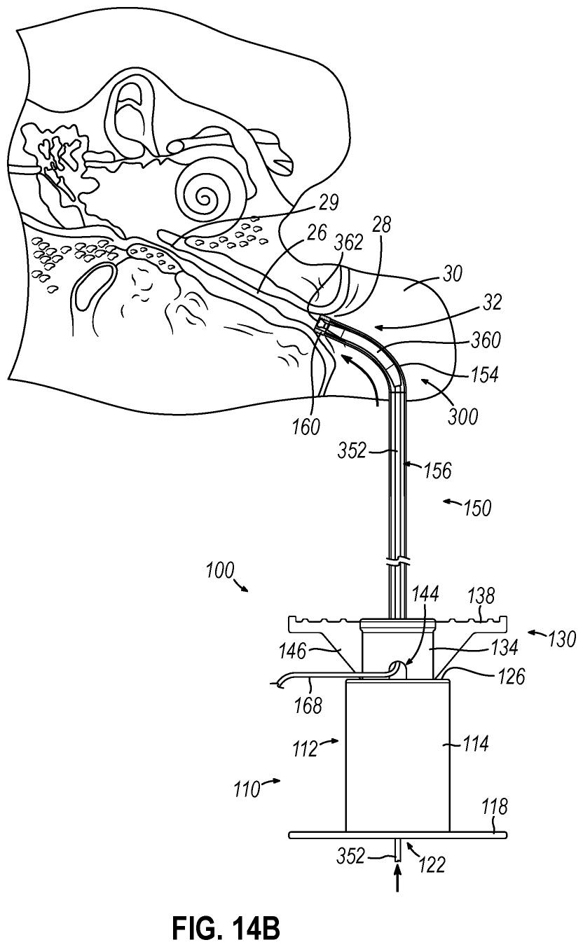

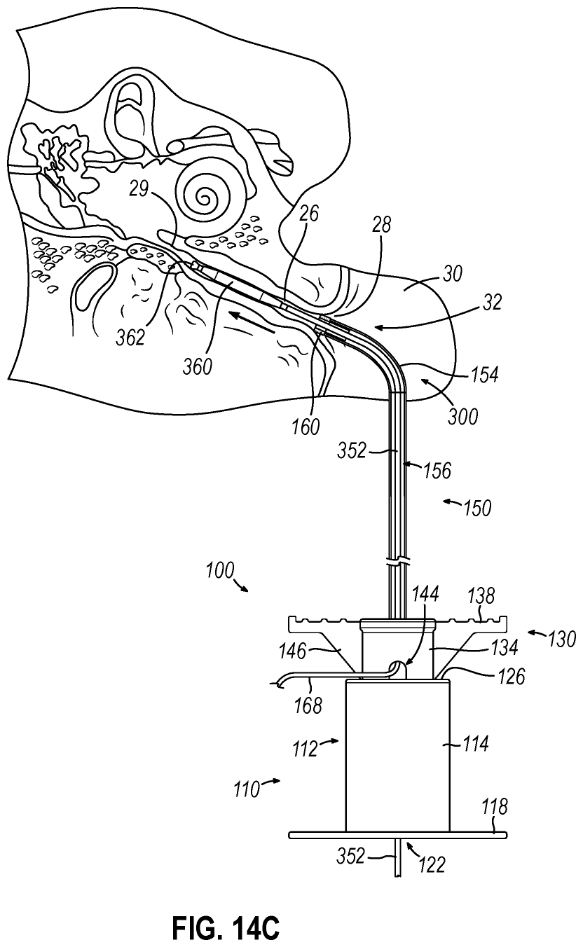

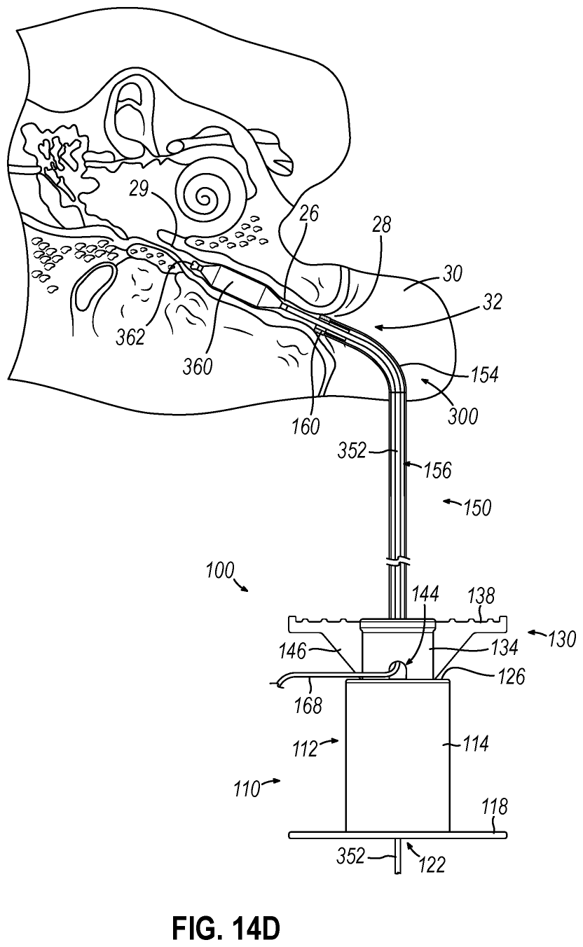

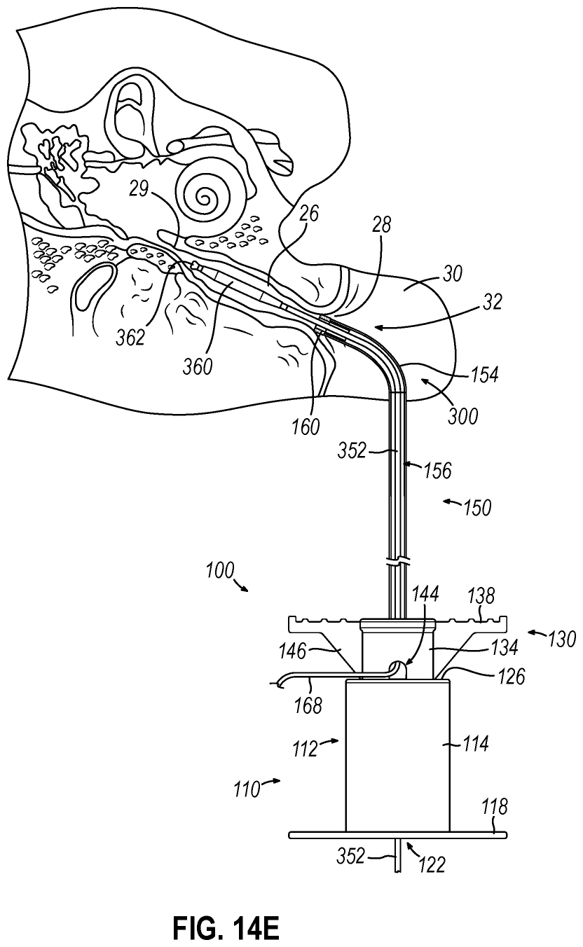

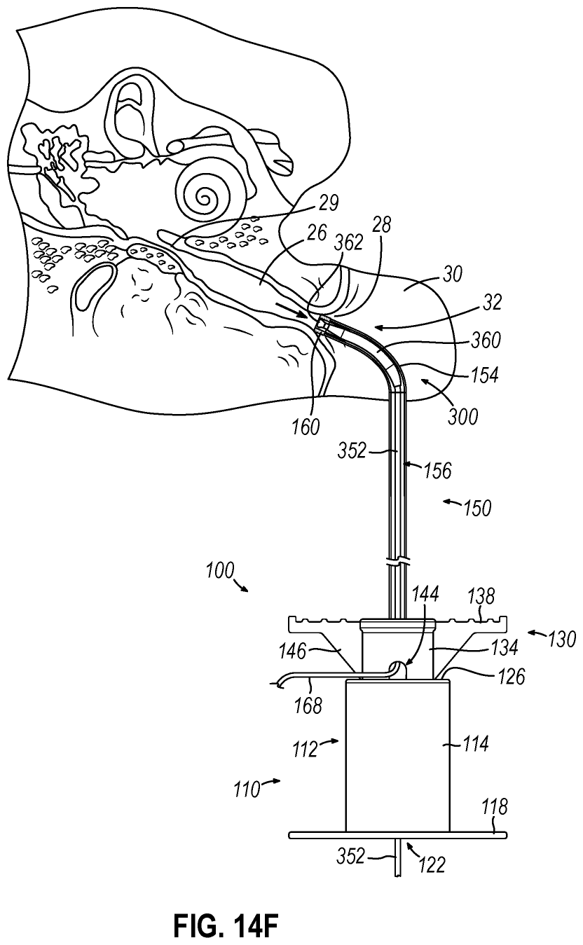

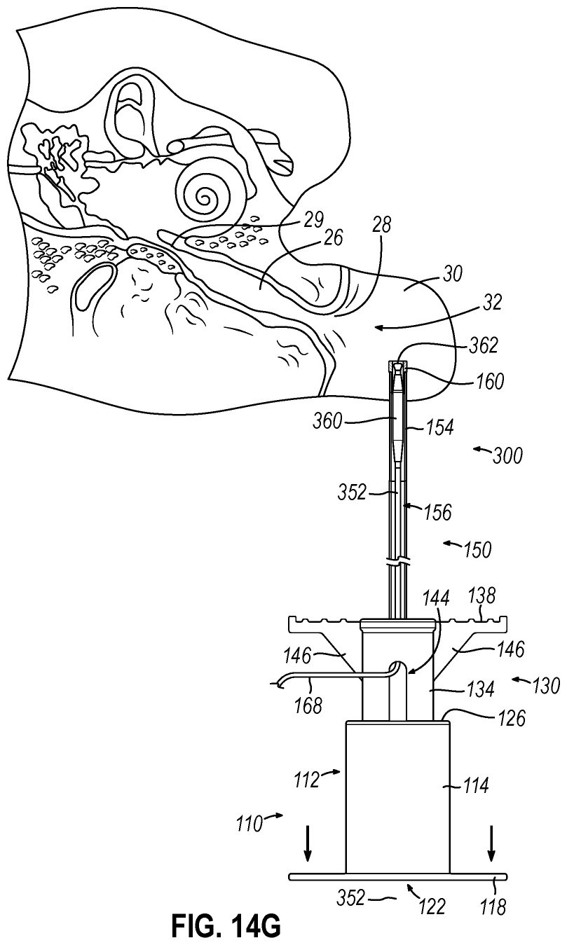

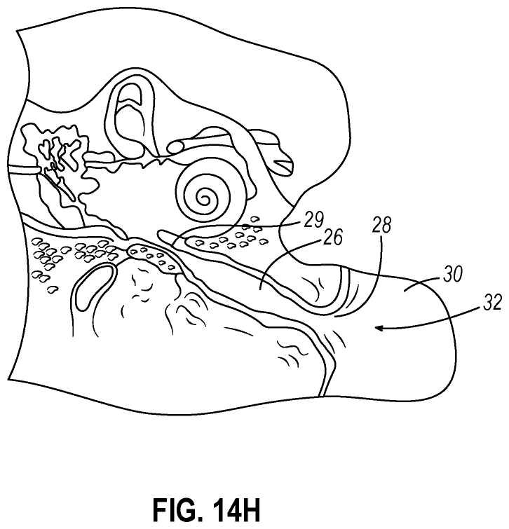

The collar 120 is dimensioned to couple with a proximal end 152 of the elongate guide catheter shaft 150 such that the proximal end 152 of the guide catheter shaft 150 is fixed relative to the proximal handle section 112. The proximal end 152 of the guide catheter shaft 150 and the collar 120 may couple with each other using any suitable means as would be apparent to one skilled in the art in view of the teachings herein. The collar 120 also defines the lumen 122. As shown in FIGS. 6A-6B, the lumen 122 includes an open proximal end defined by the flange 118 and an open distal end defined by the collar 120. Therefore, the lumen 122 is configured to slidably receive an elongate shaft 352 (see FIGS. 9, 14A-14G) of a dilation catheter 300 that may be used in conjunction with the guide catheter 100 to suitably dilate an ET 26 of a pediatric patient in accordance with the description herein.

Turning back to FIG. 4, a distal end of the proximal handle section 112 defines an opening in communication with the internal chamber 115. The opening is dimensioned to slidably receive select portions of the distal handle section 130. Additionally, at least one, or a plurality, of the protrusions 124 extend from an inner surface of the proximal handle section 112 defining the internal chamber 115. The protrusions 124 are dimensioned to slidably fit within a corresponding elongate slot 144 defined by the distal handle section 130. Interaction between the elongate slot 144 and the protrusion 124 rotationally aligns the proximal handle section 112 and the distal handle section 130. Optionally, a proximal end of at least one the elongate slots 144 may be configured to engage a corresponding protrusion 124 in order to inhibit the portions 112, 130 from disassociating with each other.

In the current example, the elongate slot 144 extends along a substantially linear profile. However, in other examples, the elongate slot 144 may extend along a suitable length of the distal handle section 130 with other suitable profiles. For example, the elongate slot 144 may extend in curved fashion along the distal handle section 130 with various distally presented surfaces. Such distally presented surfaces may engage with the protrusions 124 in order to help “lock” the longitudinal position of the distal handle section 130 relative to the proximal handle section 112. Such “locking” may help maintain an articulated configuration (see FIG. 6B) of the shaft 150 without requiring the clinician to overcome the bias force of the spring 102. In other words, the profile of the elongate slot 144, or other features, may be utilized in order to keep the shaft 150 in various deflected configurations without the assistance of the clinician squeezing the handle sections 112, 130 together to constantly overcome the bias force of the spring 102.

The distal end of the proximal handle section 112 also includes a distally presented surface 126. As will be described in greater detail below, the distally presented surface 126 is configured to abut against a hard stop 146 of the distal handle section 130, which thereby inhibits accidental damaging of the cable sleeve 168 (see FIG. 3) as the sections 112, 130 are squeezed toward each other.

The distal handle section 130 includes a first body 134 and a second body 136 that mate together in order to define an internal chamber 135. The first body 134 includes a distal flange 138 defining an opening 140 that receives the proximal rigid section 151 of the elongate guide catheter shaft 150. The distal flange 138 of the distal handle section 130 and the proximal flange 118 of the proximal handle section 112 are dimensioned to allow a clinician to grasp and control the handle assembly 110 with their index/ring finger and thumb, respectively. Therefore, the flanges 118, 138 promote the ability for a clinician to grasp and control the guide catheter 100 with a single hand; including the ability to selectively deflect the shaft 150 between the substantially straight configuration (see FIGS. 3, 5, and 6A) and the bent configuration (see FIG. 6B).

The internal chamber 135 is dimensioned to house a section of the shaft 150. As mentioned above, the distal handle section 130 defines an elongate slot 144. A cable sleeve 168 extends from the section of the shaft 150 housed within the internal chamber 135, through the elongate slot 144, and terminates into communication wires 163, 167 and their respective connectors 108, 106. As will be described in greater detail below, the connectors 108, 106 are configured to couple with a visual display and a light box connector in order to promote visualization of anatomical structures during illustrative use of the guide catheter 100 in accordance with the description herein.

Additionally, a pull wire anchor 172 (see FIGS. 6A-6B) is fixed to the interior surface defining the internal chamber 135. The pull wire anchor 172 attaches a proximal section of a pull wire 170 to the distal handle section 130 such that, as handle portions 112, 130 are actuated towards each other, the pull wire 170 moves relative to the proximal rigid section 151 of the shaft 150 to thereby deflect a deflectable distal portion 154 of the shaft 150 in accordance with the description herein.

The distal handle section 130 terminates proximally into a proximally presented surface 132. The proximally presented surface 132 is engaged with the spacer 104 such that the bias spring 102 biases the distal handle section 130 distally relative to the proximal handle section 112. In the current example, a spacer 104 and the spring 102 are used to bias the distal handle section 130 distally away from the proximal handle portion 112. However, it should be understood that any suitable biasing means may be utilized as would be apparent to one skilled in the art in view of the teaching herein.

The distal handle section 130 also includes one or more the hard stops 146 protruding outwardly from an outer surface of the at least one body 134, 136. As mentioned above, the hard stops 146 are dimensioned to abut against the distally presented surface 126 once the deflectable distal portion 154 of the shaft 150 reaches is fully articulated configuration (see FIG. 6B). The hard stops 146 are dimensioned to abut against the distally presented surface 126 of the proximal handle section 112 such that a portion of the elongate slot 144 remains distal relative to the surface 126. As shown in FIG. 6B, the portion of the elongate slot 144 extends distally relative to the surface 126 while the handle sections 112, 130 provide clearance for the cable sleeve 168 to extend out of the slot 144 while the handle sections 112, 130 are squeezed together. In other words, the hard stop 126 ensures that the cable sleeve 168 does not get crushed, or otherwise damaged, as handle sections 112, 130 acuate toward each other to deflect the shaft 150 in accordance with the description herein.

As best shown in FIGS. 3-6B, the elongate guide catheter shaft 150 includes a proximal rigid section 151 extending distally from the handle assembly 110, a deflectable distal portion 154 located distal relative to the proximal rigid section 151, and a visualizing distal tip 160. The visualizing distal tip 160 is attached to the distal end of the deflection distal portion 154. The elongate guide catheter shaft 150 defines a working lumen 156 that extends between the proximal end 152 all the way through the distal tip 160. As best shown in FIGS. 6A-6B, the lumen 156 is in communication with the opening defined by the proximal flange 118, thereby allowing the dilation catheter 300 (see FIG. 9) to be fed through the proximal end of the handle assembly 110 and into the working lumen 156. Additionally, the working lumen 156 is sufficiently large enough to allow the dilation catheter 300 to slidably attach to catheter the shaft 150 to allow actuation of the dilator 360 distally past the tip 160 into ET 26 during illustrative use in accordance with the description herein.

The visualizing distal tip 160, the deflectable distal portion 154, and appropriate sections of the proximal rigid section 151 are dimensioned to be inserted transnasally within a pediatric patient. The deflectable distal portion 154 has sufficient column strength to maintain structural integrity while being inserted transnasally within the pediatric patient to facilitate access to the ET 26. In particular, the deflectable distal portion 154 may be inserted transnasally within a pediatric patient while in the substantially straight configuration as exemplified in FIGS. 3, 5, and 6A. As mentioned above, the anatomical structures of a pediatric patient may be substantially smaller than those of an adult patient. Therefore, being able to insert the deflectable distal portion 154 while in the straight configuration may allow for transnasal insertion/retraction into and out of a pediatric patient without undesirably damaging adjacent anatomical structures.

The deflectable distal portion 154 is configured to bend relative to the long axis of proximal rigid section, as shown between FIG. 6A-6B, in accordance with the teachings herein. The deflectable distal portion 154 is formed of a suitable material to bend relative to the long axis of the proximal rigid section 151 while also maintaining a sufficient column strength, in accordance with the teachings herein. The deflectable distal portion 154 may be formed of any suitable material as would be apparent to one skilled in the art in view of the teachings herein. For example, the deflectable distal portion 154 may be formed of a braided mesh-like material having memory shape qualities. As another example, the deflectable distal portion 154 could be formed of a coiled and/or laser cut metallic material, such as nitinol, etc.

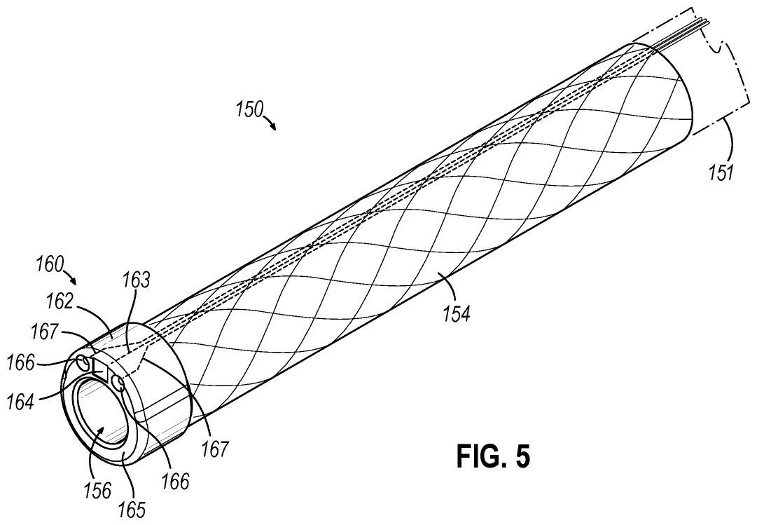

Turning to FIG. 5, the visualizing distal tip 160 includes an annular frame 162 having a distally presented annular surface 165. A camera 164 and two light sources 166 are housed within the annular frame 162 and facing outward from the distally presented annular surface 165 such that the camera 164 captures images of objects located distally from the visualizing tip 160 and such that the light sources 166 emit light to illuminate objects in the images captured by the camera 164. In some instances, the annular frame 162 is configured to spread and/or diffuse the beam of illuminated light emitted from the light sources 166.

A camera communication wire 163 and a pair of light communication wires 167 extend proximally along the deflectable distal portion 154 and the proximal rigid section 151 of the guide catheter shaft 150. The wires 163, 167 extend away from the shaft 150 into the internal chamber 145, where the wires 163, 167 may be collectively contained by the cable sleeve 168. The cable sleeve 168 extends out of the slot 144 a suitable distance in order to organize the wires 163, 167, which eventually diverge out of the cable sleeve 168 to terminate into a respective connector 108, 106.

The connector 106 is in communication with the light communication wires 167. Additionally, the connector 106 is configured to couple with a light box, such that light box may generate light, and communicate such light to the light sources 166 at the distal tip 160 via the connector 106 and the wires 167. The connector 108 is in communication with the camera communication wire 163. Additionally, the connector 108 is configured to couple to a visual display such that images captured by the camera 164 may be displayed on the visual display via the wire 163 and the connector 108. Therefore, during illustrative use in accordance with the description herein, the connectors 106, 108 are operatively connected to devices that enable functionality of the light sources 166 and the camera 164.

The camera 164 may have any suitable field of view as would be apparent to one skilled in the art in view of the teachings herein. Additionally, the camera 164 may include any suitable components as would be apparent to one skilled in the art in view of the teachings herein. Light sources may include an LED, an optical fiber, or any other suitable source of light as would be apparent to one skilled in the art in view of the teachings herein.

In some instances, the distal tip 160 may also have a position sensor configured to interact with a suitable navigation system to track the display the location of the distal tip 160 during illustrative use in accordance with the description herein. There could also be a position sensor located at a proximal end of the deflectable shaft portion 154. Having a position sensor located at the distal tip 160 and the proximal end of the deflectable shaft portion 154 may provide better positional data on the bend angle and/or pose of the shaft 150 and/or the deflectable shaft portion 154. By way of example only, such a position sensor may take the form of one or more coils that generate signals in response to an alternating electromagnetic field, where those signals indicate the real-time location of the position sensor in three-dimensional space.

As mentioned above, a pull wire 170 is attached to the guide catheter shaft 150. As best shown in FIG. 6A-6B, a distal end of the pull wire 170 is fixed to a distal portion of either the deflectable distal portion 154 or the distal tip 160. The pull wire 170 extends proximally from the deflectable distal portion 154 and connects to the pull wire anchor 172. The pull wire 170 may be interposed between an inner diameter of the shaft 150 defining the working lumen 156 and an outer diameter the shaft 150. In some instances, the pull wire 170 may extend within the inner diameter of the shaft 150. Yet in other instances, the pull wire 170 may extend along the outer diameter of the shaft 150. Therefore, it should be understood that the pull wire 170 may extend adjacent to any suitable portion of the shaft 150 as would be apparent to one skilled in the art in view of the teachings herein. As mentioned above, the pull wire 170 actuates relative to the proximal rigid section 151 in response to a clinician moving the handle sections 112, 130 relative to each other, thereby driving deflection of the deflectable distal portion 154 in accordance with the description herein.

As shown in FIGS. 6A-6B, a clinician may squeeze on the flanges 118, 138 with their thumb and index/ring finger in order to overcome the biasing force of the spring 102, thereby compressing the spring 102. Since the proximal end of the pull wire 170 is attached to the pull wire anchor 172, which is fixed to the distal handle section 130, and since the proximal rigid section 151 of the shaft 150 is fixed to the proximal handle section 112, movement of the handle sections 112, 130 relative to each other drives translation of the pull wire 170 relative to the proximal rigid section 151 of the shaft 150. Since the distal end of the pull wire 170 is fixed to relative to a distal portion of either the deflectable distal portion 154 or the distal tip 160, such actuation of the pull wire 170 drives deflection of the deflectable distal portion 154. Therefore, a clinician may control the degree of deflection of the deflectable distal portion 154 by utilizing a single hand grasping the handle assembly 110. The deflectable distal portion 154 is configured to deflect relative to the longitudinal axis of the proximal rigid section 151 to define a suitable angle configured to facilitate access to the ET 26 of a pediatric patient.

Conversely, if the clinician desires to bend the deflectable distal portion 154 toward the substantially straight configuration, the clinician may acuate the handle sections 112, 130 in the opposite direction, by either moving the handle sections 112, 130 away from each other or allowing the bias spring 102 to drive the handle sections 112, 130 away from each other. The deflectable distal portion 154 is biased toward the straight configuration. Therefore, as the handle sections 112, 130 move back toward the position shown in FIG. 6A, the pull wire 170 releases tension on either the distal portion of either the deflectable distal portion 154 or the distal tip 160, thereby allowing the resilient nature of the deflectable distal portion 154 to return toward the substantially straight configuration. Therefore, it should be understood that a clinician may control the deflection angle of the deflectable distal portion 154 with a single hand grasping the handle assembly 110. As will be described in greater detail below, the clinician may manipulate the degree of deflection of the distal tip 160 and the deflectable distal portion 154 in order to facilitate access to the ET 26 of a pediatric patient.

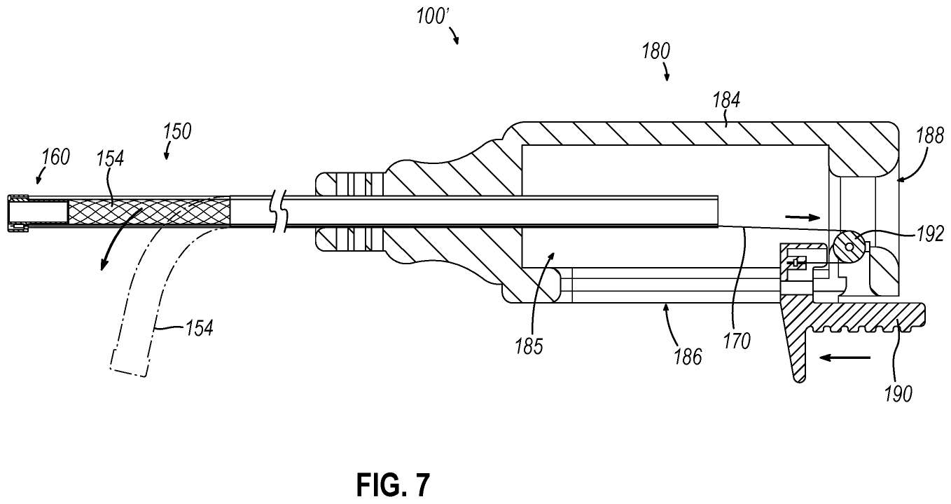

While the pull wire 170, the pull wire anchor 172, the bias spring 102, and the handle sections 112, 130 are used in the current example to drive deflection of the deflectable distal portion 154, any other suitable mechanisms may be utilized as would be apparent to one skilled in the art in view of the teachings herein. FIG. 7 shows an alternative deflectable guide catheter 100′ that may be used in replacement of the deflectable guide catheter 100 described herein. Therefore, the deflectable guide catheter 100′ is substantially similar to the deflectable guide catheter 100 described herein, with differences described below. The deflectable guide catheter 100′ includes the elongate guide catheter shaft 150 and the visualizing distal tip 160.

However, the deflectable guide catheter 100′ includes an alternative handle assembly 180. The handle assembly 180 includes a body 184, a slider 190, and a pulley 192. The body 184 is configured to be grasped by a single hand of a clinician while the slider 190 may be actuated relative to the body 184 with the same hand grasping the body 184. The body 184 defines an internal chamber 185 which houses a proximal portion of the shaft 150 such that rigid proximal section (151) is fixed to the body 184. The body 184 also defines a proximal opening (188) which is dimensioned to slidably receive a shaft assembly 350 of the dilation catheter 300 (see FIG. 9). Therefore, the dilation catheter 300 (see FIG. 9) may be fed through a proximal portion of the handle assembly 100′ into the working lumen 156 of the shaft 150 for illustrative use in accordance with the description herein.

The body 184 also defines an elongate slot 186 which the slider 190 is slidably disposed within. A proximal end of the pull wire 170 loops around a pulley 192 that is fixed to the body 184. The proximal end of the pull wire 170 is fixed to the slider 190 such that movement of the slider 190 relative to the body 184 along the path defined by the elongate slot 186 drives movement of the pull wire 170. In particular, a clinician may actuate the slider 190 distally in order to drive the deflectable distal portion 154 into a deflected configuration. Additionally, the clinician may actuate the slider 190 proximally to release tension within the pull wire 170 in order to allow the deflectable distal portion 154 to bend back toward the substantially straight configuration. Therefore, a clinician may control the position of the slider 190 in order to control the deflection angle of the deflectable distal portion 154.

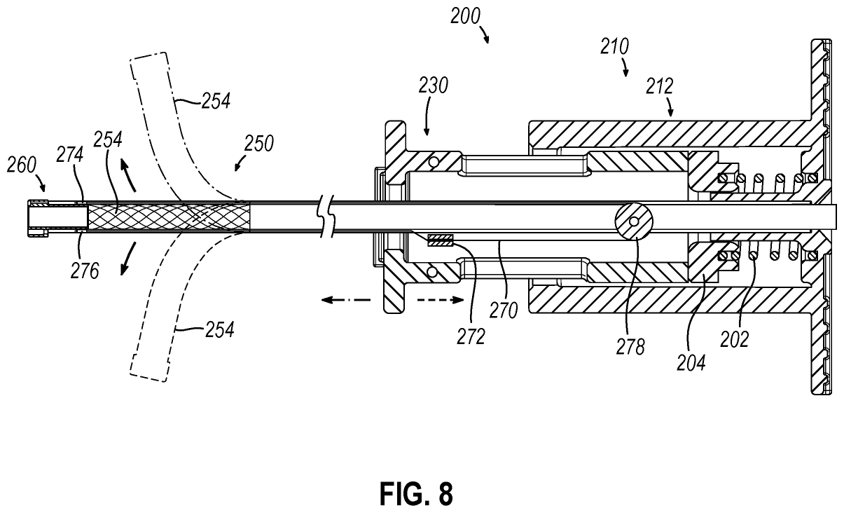

FIG. 8 shows an alternative deflectable guide catheter 200 that may be used in replacement of the deflectable guide catheter 100, 100′ described herein. Therefore, the deflectable guide catheter 200 is substantially similar to the deflectable guide catheter 100 described herein, with differences described below. The deflectable guide catheter 200 includes a spring 202, a spacer 204, a handle assembly 210, a proximal handle section 212, a distal handle section 230, an elongate guide catheter shaft 250, and a visualizing distal tip 260, which may be substantially similar to includes the spring 102, the spacer 104, the handle assembly 110, the proximal handle section 112, the distal handle section 130, the elongate guide catheter shaft 150, and the visualizing distal tip 160 described above, with differences elaborated herein.

The deflectable guide catheter 200 also includes a pull wire 270 that is fixed to a distal portion of the deflectable portion 254 and/or the distal tip 260 at a first distal section 274 and a second distal section 276. The first distal section 274 of the pull wire 270 extends proximally along the shaft 250 and is attached to a pull wire anchor 272 which is fixed to the distal handle section 230. However, rather than terminating into the anchor 272, the pull wire 270 extends proximally past the anchor 272 and loops around a pulley 278, which is fixed to the proximal handle section 212. The pull wire 270 loops around the pulley 278 and extends through the shaft 250 into the second distal section 276. Therefore, a clinician may deflect the distal portion 254 in a first direction in response to proximally actuating the distal handle section 230 relative to the proximal handle section 212. Additionally, the clinician may deflect the distal portion 254 in a second, opposite, direction in response to distally actuating the distal handle section 230 relative to the proximal handle section 212.

While the pulley 278 is used to redirect the pull wire 270 between the first distal section 274 and the second distal section 276, any other suitable structures may be utilized as would be apparent to one skilled in the art in view of the teaching herein. For example, a U-shaped track or hypotube may be utilized to redirect the pull wire 270 between the sections 274, 276 of the pull wire 270.

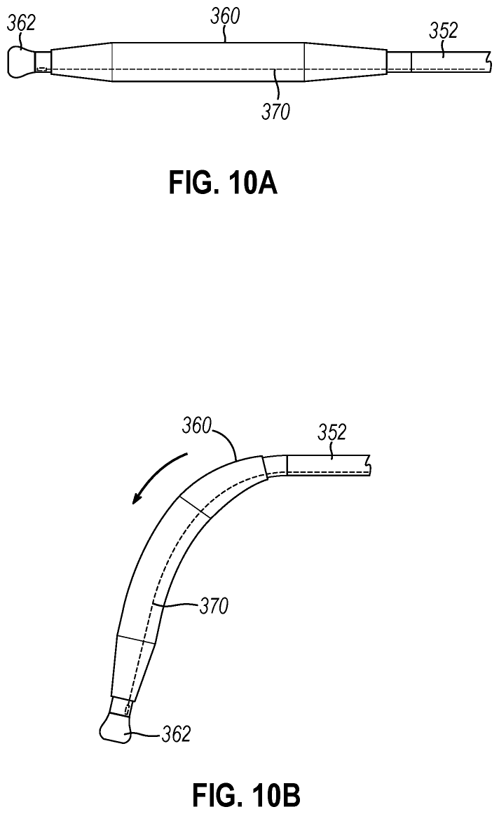

FIG. 9 shows an illustrative deflectable dilation catheter 300 that may be utilized in conjunction with, or in the absence of, the deflectable guide catheter 100, 100′, 200 described above in order to dilate the ET 26 of pediatric patient. As will be described in greater detail below, the deflectable dilation catheter 300 is also configured to be selectively deflected between a substantially straight configuration (see FIG. 10A) and a bent configuration (see FIG. 10B). While some examples described below include use of the deflectable dilation catheter 300 with the deflectable guide catheter 100, 100′, 200, it should be understood that the deflectable dilation catheter 300 may be used without the deflectable guide catheter 100, 100′, 200. For instance, the dilation catheter 300 may be used without any guide catheter at all in some cases. As another example, the dilation catheter 300 may be used with a non-deflectable guide catheter in some cases. In some such scenarios, the dilation catheter 300 may be inserted through a substantially straight catheter until the deflectable region of the dilation catheter 300 protrudes distally from the open distal end of the non-deflectable guide catheter; and then the deflectable region of the dilation catheter 300 may be deflected toward the ET 26 to then enter the ET 26. In some scenarios where the dilation catheter 300 is used with a non-deflectable guide catheter, the distal region of the non-deflectable is substantially straight or otherwise lacks a preformed, rigid the bend 94 like the guide catheter 90.

The deflectable dilation catheter 300 includes a handle assembly 310, a shaft assembly 350, a dilator in the form of a balloon 360, and a navigation system connector 380 extending proximally from the handle assembly 310. The handle assembly 310 includes a body 312 defining an elongate slot 314 and an actuator 316 slidably coupled to the body 312 via the elongate slot 314. The body 312 is dimensioned to be grasped by a single hand of a clinician such that the clinician may also control the actuator 316 with the same hand. The body 312 defines a distal recess 318 dimensioned to house a portion of the rotation knob 355 of the shaft assembly 350. The distal recess 318 may house a portion of the rotation knob 355 at multiple, discrete, angular positions; thereby locking the shaft assembly 350 into a corresponding angular position relative to the handle assembly 310. Engagement between the distal recess 318 and the rotation knob 355 allows a clinician to control the orientation of the balloon 360 about its own long axis relative to the body 312. Controlling the orientation of the balloon 360 about its own long axis may allow a clinician to use a single deflectable dilation catheter 300 to access either the right or left ET 26 of a patient, while controlling the dilation catheter 300 with the same hand.

The actuator 316 is configured to slide relative to the body 312 along a path defined by the elongate slot 314. As will be described in greater detail below, the actuator 316 is attached to a pull wire 370 such that movement of the actuator 316 relative to the body 312 is configured to bend the shaft assembly 350 and the balloon 360 between the substantially straight configuration (see FIG. 10A) and the bent configuration (see FIG. 10B).

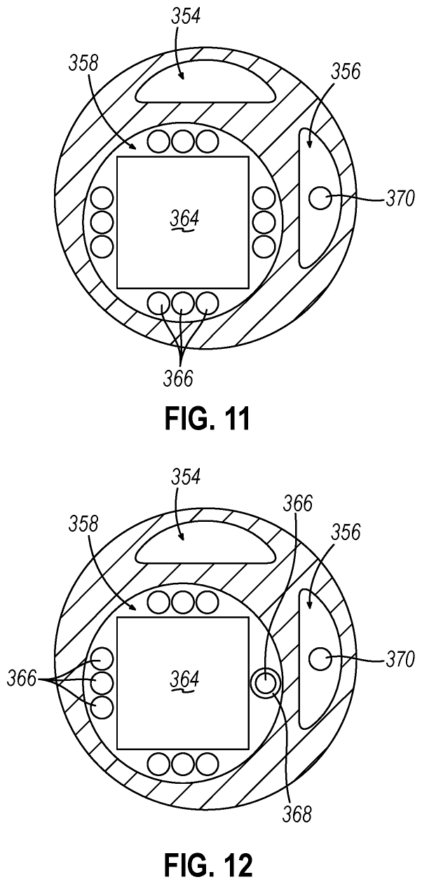

A luer fitting 320 extends from the body 312. The luer fitting 320 is configured to selectively couple with a suitable source of fluid (i.e., saline, air, etc.). The luer fitting 320 is in fluid communication with an inflation lumen 354 (see FIG. 11) of the shaft assembly 350, which terminates distally into communication with the interior of the balloon 360. Therefore, the luer fitting 320 and the inflation lumen 354 are configured to provide fluid communication between a suitable source of fluid and the interior of the balloon 360 in order to selectively inflate and deflate the balloon 360 in accordance with the description herein.

The shaft assembly 350 includes an elongate shaft 352 extending distally from the handle assembly 310 into a distal tip 362 and a rotation knob 355 fixed to the elongate shaft 352. The elongate shaft 352 may be substantially flexible in order to be advanced and retracted within the working lumen 356 of the elongate guide catheter shaft 150 in order to suitably access the ET 26 of a pediatric patient in accordance with the description herein. Additionally, the elongate shaft 352 has sufficient column strength such in order to actuate the distal tip 362 into an obstructed and/or blocked ET 26 without buckling. Therefore, appropriate sections of the elongate shaft 352 may be configured to bend in response to being advanced through the deflectable distal portion 154 of the elongate guide catheter shaft 150 in the bent configuration (see FIGS. 14B-14C).

In the current example, the distal tip 362 is a bulbous polymeric blueberry-shaped, atraumatic tip. The smoothness and roundness of the tip 362 facilitates advancement of the balloon catheter 300 by helping it glide smoothly through the ET 26. The tip 362 further acts as a safety stop. The diameter of the tip 362 is larger than the outer diameter of the elongate shaft 352 such that the tip 362 will prevent the balloon catheter 300 from passing through the isthmus 29 into the middle ear 14. In some instances, the distal tip 362 may have a position sensor configured to be used with a suitable navigation system in order to track and display the position of the distal tip 362. The position sensor may be integrated into the distal tip 362 itself or may be fixed relative to distal tip. In some instances, the position sensor may be incorporated into a guide wire, as described below. A position sensor on the distal tip 362 may be used on conjunction with position sensor on the shaft 150 (the position sensors at the distal tip 160 or the position sensor at proximal end of the deflectable distal portion 154) in order to provide better positional data on the bend/pose of the elongate shaft 352. By way of example only, a position sensor at the distal tip 362 may take the form of one or more coils that generate signals in response to an alternating electromagnetic field, where those signals indicate the real-time location of the position sensor in three-dimensional space.

As shown in FIG. 11, the elongate shaft 352 defines the inflation lumen 354, a pull wire lumen 356, and a working lumen 358. As mentioned above, the inflation lumen 354 extends between the interior of the balloon 360 and the luer fitting 320 in order to communicate suitable fluid into and out of the balloon 360 for inflation/deflation purposes. The pull wire lumen 356 slidably receives the pull wire 370, which extends from a distal portion of the balloon 360 all the way to the actuator 316 of the handle assembly 310. The pull wire 370 is fixed to the distal portion of the balloon 360 as well as the actuator 316. Therefore, a clinician may slide the actuator 316 along the path defined by the elongate slot 314 in order to acuate the pull wire 370 within the pull wire lumen 356, thereby deflecting the balloon 360 between the substantially straight configuration (see FIG. 10A) and the bent configuration (see FIG. 10B). The balloon 360 may be resiliently biased toward the straight configuration such that once tension from the pull wire 370 is relieved, the balloon 360 may bend itself back toward the straight configuration.

The working lumen 358 extends through the elongate shaft 352 all the way to an open distal tip 362. In some instances, the working lumen 358 and the open distal tip 362 may allow for pressure ventilation between ends of the ET 26 as the balloon 360 is positioned within ET 26 in accordance with the description herein. The working lumen 358 may house suitable components as would be apparent to one skilled in the art in view of the teachings herein. For example, as shown in FIG. 11, the working lumen 358 may house a camera 364 and a plurality of light sources 366, which may be located near the open distal tip 362. The camera 364 and the light sources 366 can be utilized to visualize suitable objects. In some instances, the camera 364 and the light sources 366 may be fixed within the working lumen 358 and adjacent to the open distal tip 362 in order to view objects located distally from the open distal tip 362. The camera 364 may therefore be utilized to reach and visualize anatomies that would be otherwise difficult to view with a traditional endoscope 60.

In other instances, the camera 364 and the light sources 366 (e.g., light fibers, LEDs, etc.) may be slidably contained within the working lumen 358 such that the camera 364 and the light sources 366 may be advanced and retracted relative to the working lumen 358 as a unit. In such instances, for example, a clinician may maintain the position of the camera 364 and the light source 366 relative to anatomy as the balloon 360 is advanced and retracted relative to the ET 26 of the pediatric patient. In such instances, a clinician may utilize the camera 364 in order to visualize indicators on the balloon 360 as the balloon 360 is actuated within the ET 26. Such visual indicators may indicate how far the balloon 360 has been actuated out of the deflectable distal portion 154 of the guide catheter 100 during illustrative use in accordance with the description herein.

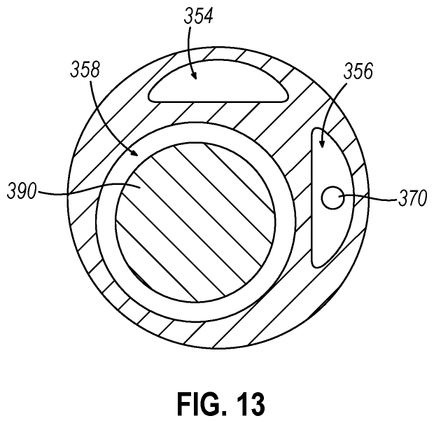

In some instances, as shown in FIG. 12, a position sensor 368 may be attached to and concentric with a light source 366 (e.g., light fiber), where the camera 364, the light sources 366, and the position sensor 368 are configured to move relative to the working lumen 358 as a unit. Such a position sensor 368 may be detected and tracked by a suitable navigation system as described herein. Such a navigation system may superimpose the detected and tracked position of the position sensor 368 relative to the one or more images of the patient's anatomy shown on a visual system. Additionally, having the position sensor 368 and the camera 364 configured to actuate as a unit may allow for various other functionalities, such as augmented reality features.

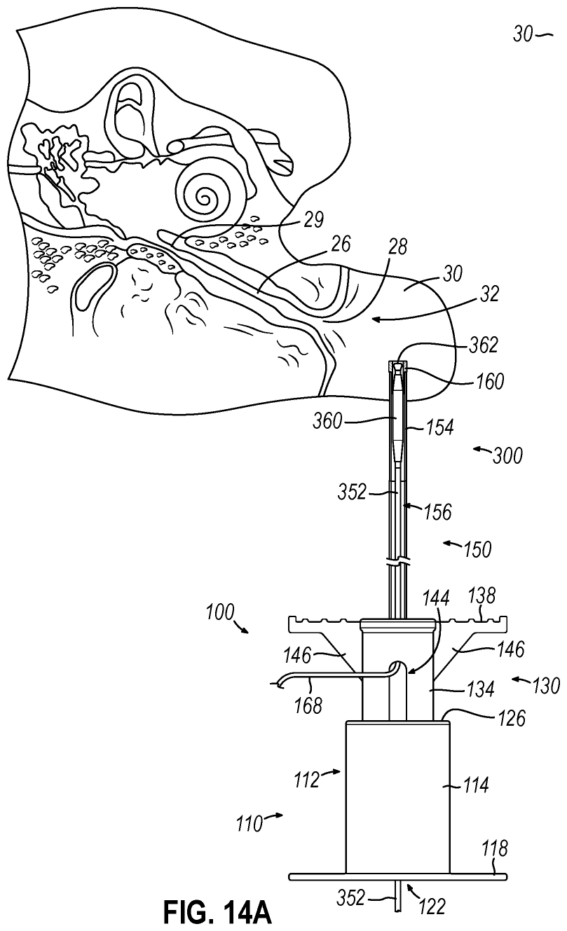

In some instances, as shown in FIG. 13, just a navigation wire 390 is attached to the working lumen 358. Such a navigation wire 390 may include one or more position sensors as described herein, allowing the navigation wire 390 to be detected and tracked by a suitable navigation system. Such a navigation system may superimpose the detected and tracked position of the navigation wire 390 relative to one or more images the patient's anatomy shown on a visual system.

As mentioned above, the navigation system connector 380 extends from the handle assembly 310. Suitable communication wires may extend from the camera 364, the light sources 366, the position sensor 368, and/or the navigation wire 390, through the working lumen 358, and into the navigation system connector 380. The navigation system connector 380 may also be configured to couple with suitable electronics in order to establish communication between the camera 364, the light sources 366, and the position sensor 368, 390 with such electronics. Any suitable electronics may be utilized as would be apparent to one skilled in the art in view of the teachings herein.

FIGS. 14A-14H show an illustrative use of the deflectable guide catheter 100 and the deflectable dilation catheter 300 in order to suitably access, dilate, and withdraw from the ET 26 of a pediatric patient. While the deflectable guide catheter 100 is used in the current example, it should be understood that the deflectable guide catheter 100′, 200 may also be used in replacement of the guide catheter 100. It should also be understood that a non-deflectable dilation catheter may be used with the deflectable guide catheter 100, 100′, 200 in lieu of the deflectable dilation catheter 300 being used with the deflectable guide catheter 100, 100′, 200. Moreover, a non-deflectable guide catheter may be used with the deflectable dilation catheter 300 in lieu of the deflectable guide catheter 100, 100′, 200 being used with the deflectable dilation catheter 300.

First, as shown in FIG. 14A, the deflectable dilation catheter 300 may be preloaded into the deflectable guide catheter 100. The balloon 360 and suitable portions of the elongate shaft 350 may be inserted into the lumen 122 of the collar 120 via the proximal flange 118, and further advanced into the working lumen 156 of the elongate guide catheter shaft 150. In some instances, as shown in FIG. 14A, the balloon 360 may be advanced distally within the working lumen 156 until the balloon 360 is directly adjacent to the deflectable distal portion 154 of the elongate guide catheter shaft 150. While not shown, it should be understood that the handle assembly 310 of the dilation catheter 300 may be located proximally relative to the proximal flange 118 of the guide catheter 100. In some instances, the handle assemblies 310, 110 may be controlled by the same hand of a clinician, either simultaneously or sequentially.

With the balloon 360 preloaded within the working lumen 156, the deflection distal portion 154 and the distal tip 160 may be advanced transnasally into the pediatric patient while in the straight configuration, as also shown in FIG. 14A. The clinician may also utilize the camera 164 and the light sources 166 of the visualizing distal tip 160 (or the camera 364 and the light sources 366 of the dilation catheter 300) in order to visually confirm the deflectable distal portion 154 and the distal tip 160 are suitably positioned within the nasal cavity. As mentioned above, since anatomical features of a pediatric patient may be substantially smaller than those of an adult patient, being able to insert the deflectable distal portion 154 while in the straight configuration may facilitate transnasal insertion/retraction into and out of a pediatric patient without undesirably damaging adjacent anatomical structures.

Next, as shown in FIG. 14B, the clinician may bend the deflectable distal portion 154 of the guide catheter 100 and/or the balloon 360 in accordance with the description herein. In particular, clinician may bend the deflectable distal portion 154 of the guide catheter 100 unit the distal tip 160 is suitably aligned with the pharyngeal ostium 28. As mentioned above, the deflectable distal portion 154 is configured to reach a suitable angle of deflection in order to facilitate access to the ET 26 of a pediatric patient. Since the deflectable distal portion 154 may be inserted in the substantially straight configuration and then subsequently bent into the deflected configuration, the guide catheter 100 may provide access to the ET 26 of a pediatric patient without damaging adjacent anatomical structures. Again, the clinician may also utilize the camera 164 and the light sources 166 of the visualizing distal tip 160 (or the camera 364 and the light sources 366 of the dilation catheter 300) in order to visually confirm suitable placement and alignment of the distal tip 160 relative to the ET 26. It should be understood that a clinician may control the deflection angle of the deflectable distal portion 154 with the same hand that controls placement of the guide catheter 100. In instances where a locking mechanism is provided that may selectively lock the deflectable distal portion 154 in a bent configuration, the clinician may lock deflectable distal portion 154 into the bent configuration shown in FIG. 14B such that the clinician is not required to constantly overcome the biasing force of the spring 102.

Next, as shown in FIG. 14C, with the distal tip 160 suitably aligned, the clinician may advance a deflated balloon 360 out of the confines of the deflectable distal portion 154, distally past the tip 160, and into the ET 26 of the pediatric patient. The clinician may also utilize the camera 164 and the light sources 166 of the visualizing distal tip 160 (or the camera 364 and the light sources 366 of the dilation catheter 300) in order to visually confirm suitable placement of the balloon 360 within the ET 26 of the patient. Next, as shown in FIG. 14D, with the balloon 360 suitably positioned within the ET 26, the clinician may inflate the balloon 360 in accordance with the description herein in order to dilate the ET 26. After sufficiently dilating the ET 26 of the pediatric patient utilizing the balloon 360, the clinician may then transition the balloon 360 back into the deflated configuration as shown in FIG. 14E and retract the balloon 360 back within the confines of the working lumen 356 as shown in FIG. 14F.

Next, as shown in FIG. 14G, while the deflectable distal portion 154 and the distal tip 160 are within the nasopharynx region 30, the clinician may bend the deflectable distal portion 154 of the guide catheter 100 and/or the balloon 360 back toward the substantially straight configuration in accordance with the description herein. In some cases, this straightening is achieved simply by relieving tension in the pull wire 170, 370. With the deflectable distal portion 154 and the distal tip 160 back in the substantially straight configuration, the clinician then retracts the tip 160, the deflectable distal portion 154, and the balloon 360 out of the nasopharynx region 30, as shown in FIG. 14H. Since the deflectable distal portion 154 may facilitate access to the ET 26 of a pediatric patient in the bent configuration and then return back into the substantially straight configuration, the guide catheter 100 may provide access to the ET 26 of a pediatric patient and then be subsequently removed without damaging adjacent anatomical structures. It should be understood that a clinician may control the deflection angle of the deflectable distal portion 154 with the same hand that controls placement of the guide catheter 100. In instances where a locking mechanism is provided that may selectively lock the bent position of the deflectable distal portion 154, the clinician may unlock the deflectable distal portion 154 and thereby deflect distal portion 154 back into the substantially straight configuration as shown in FIG. 14G.

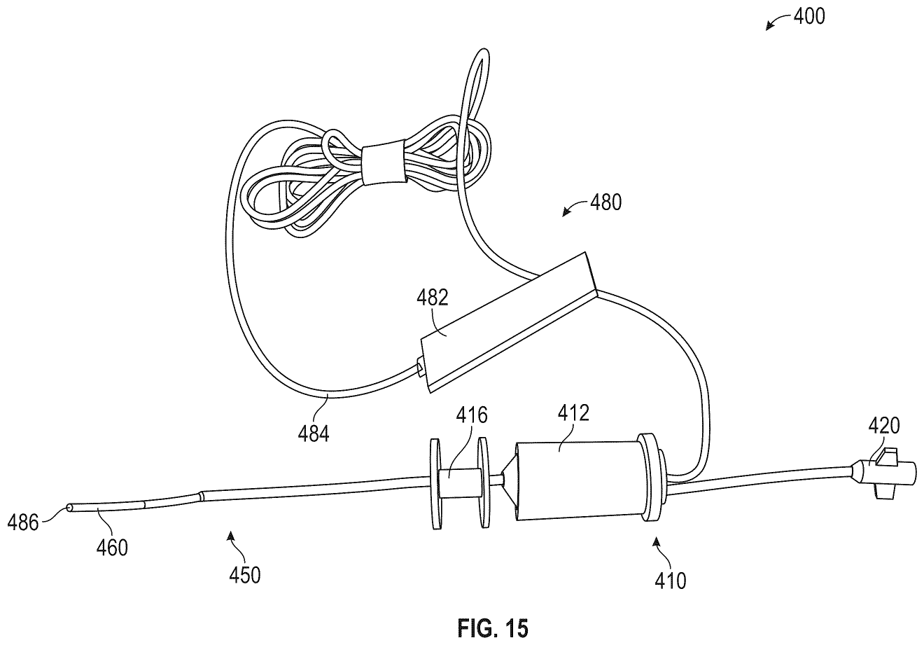

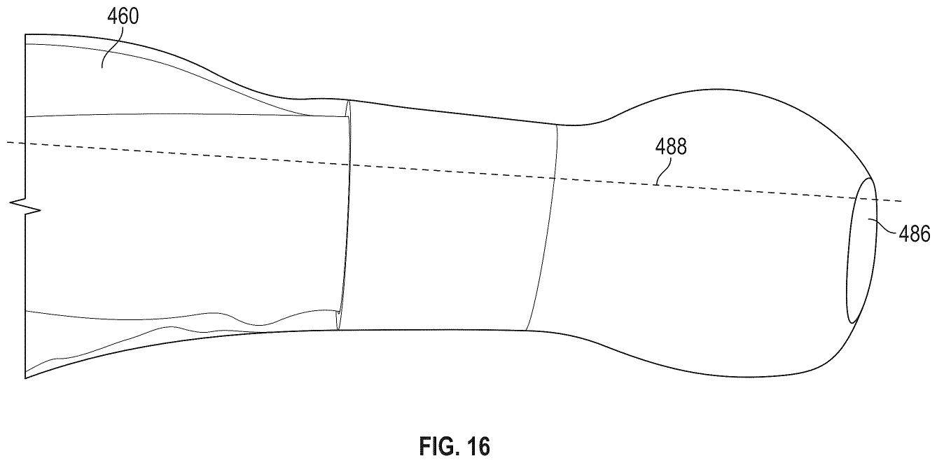

FIGS. 15-16 show an illustrative the deflectable dilation catheter 400 that may be substantially similar to dilation catheter described 300 above, with differences elaborated below. Therefore, the deflectable dilation catheter 400 may be utilized in the same scenarios contemplated with the dilation catheter 300 described herein or any other suitable scenarios as would be apparent to one skilled in the art in view of the teachings herein. The dilation catheter 400 includes a handle assembly 410, a shaft assembly 450, and a dilator in the form of a balloon 460; which may be substantially similar to the handle assembly 310, the shaft assembly 350, and the balloon 360 described above, with differences elaborated below.

The handle assembly 410 includes a body 412 configured to be grasped by a single hand of a clinician such that the clinician may also control the actuator 416 with the same hand. The actuator 416 in the current example is located distally relative to the body 412 and is slidably coupled to the body 412 via a portion of the shaft assembly 450. Similar to the actuator 316 described above, the actuator 416 is attached to the pull wire (not shown) that is substantially similar to the pull wire 370 described above. The pull wire (not shown) is configured to bend at least a distal portion of the shaft assembly 450 and the balloon 460 between a substantially straight configuration and a bent configuration.

The shaft assembly 450 defines the pull wire lumen (not shown) that is substantially similar to the pull wire lumen 356 described above. The the pull wire lumen (not shown) slidably receives the pull wire (not shown), which extends from a distal portion of the balloon 460 all the way to the actuator 416 of the handle assembly 410. The pull wire (not shown) is fixed to the distal portion of the balloon 460 as well as the actuator 416. Therefore, a clinician may slide the actuator 416 toward the body 412 along the path defined by the shaft assembly 450 in order to acuate the pull wire (not shown) within the pull wire lumen (not shown), thereby deflecting the balloon 460 between the substantially straight configuration and the bent configuration. The balloon 460 may be resiliently biased toward the straight configuration such that once tension from the pull wire (not shown) is relieved, the balloon 460 may bend itself back toward the straight configuration.

A luer fitting 420 extends from the body 412, which is substantially similar to the luer fitting 320 described above. Therefore, the luer fitting 420 is configured to selectively couple with a suitable source of fluid. Additionally, the luer fitting 420 is in fluid communication with an inflation lumen (not shown) of the shaft assembly 450 that terminates distally into communication with the interior of the balloon 460. Therefore, the luer fitting 420 and the inflation lumen (not shown) are configured to provide fluid communication between a suitable source of fluid and the interior of the balloon 460 in order to selectively inflate and deflate the balloon 460 in accordance with the description herein.

Additionally, the dilation catheter 400 includes a navigation assembly 480. The navigation assembly 480 includes a connector 482, a cable 484, a navigation sensor 486, and the electrical communication line 488. The electrical communication line 488 extends from the navigation sensor 486 all the way to the connector 482 by extending through/along the balloon 460, the shaft assembly 450, and the handle assembly 410. The cable 484 is in communication with the connectors 402. Therefore, the navigation sensor 486 is in communication with the connector 482 via the electrical communication line 488 and the cable 484. The connector 482 is configured to establish communication with a suitable surgical navigation system.

The navigation sensor 486 is fixed to a distal end of the balloon 460. The navigation sensor 486 is configured to generate a signal in response to exposure of alternative magnetic fields generated by a navigation system. Further, the navigation assembly 480 is configured to communicate that signal to a console of the navigation system via the electrical communication line 488, the cable 484, and the connector 482, thereby allowing the navigation system to determine the position of the navigation sensor 486 in three-dimensional space. Navigation system may utilize the determined position of the navigation sensor 486 in order to superimpose the position of the navigation sensor 486 onto preoperative or live images of the targeted surgical site.

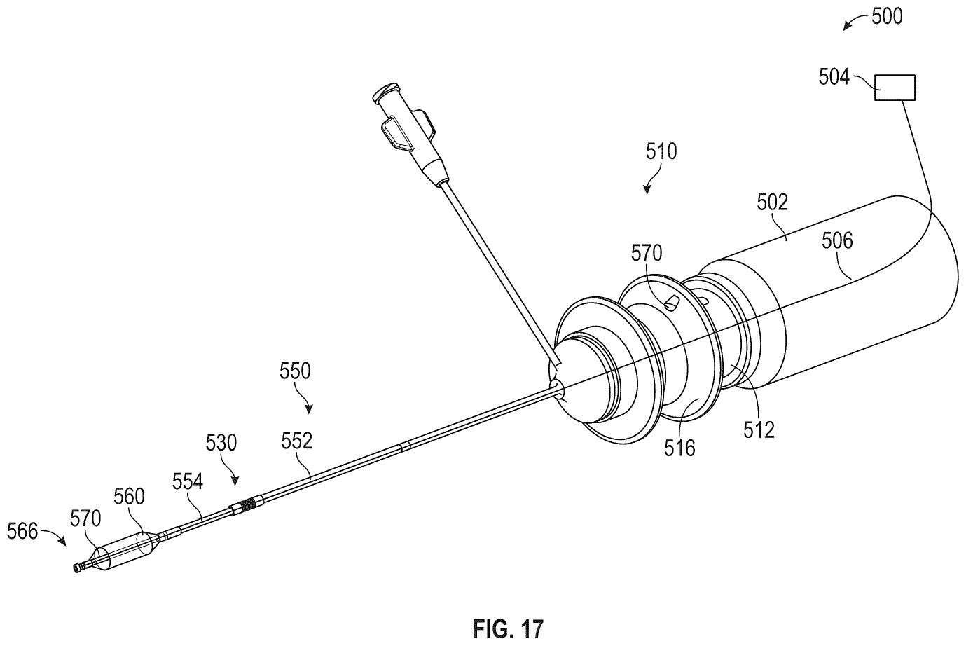

FIGS. 17-19 show an illustrative deflectable dilation catheter 500 that may be substantially similar to the dilation catheter 300, 400 described above, with differences elaborated below. Therefore, the deflectable dilation catheter 500 may be utilized in the same scenarios contemplated with the dilation catheter 300, 400 described herein or any other suitable scenarios as would be apparent to one skilled in the art in view of the teachings herein. The dilation catheter 500 includes a proximal endoscope handle 502, a handle assembly 510, a shaft assembly 550, and a dilator in the form of a balloon 560, which may be substantially similar to the handle assembly 310, 410, the shaft assembly 350, 450, and the balloon 360, 460 described above, with differences elaborated below.

The handle assembly 510 includes a body 512 configured to be grasped by a single hand of a clinician such that the clinician may also control the actuator 516 with the same hand. As shown in FIG. 17, a proximal end of the body 512 is directly coupled to a distal end of the endoscope handle 502. The endoscope handle 502 is also configured to be grasped by a clinician in order to suitably control and manipulate the deflectable dilation catheter 500 in accordance with the description herein. The endoscope handle 502 is configured to selectively couple with a viewing monitor 504 such that images captured in accordance with the description herein may be viewed by a clinician at the viewing monitor 504. As will be described in greater detail below, at least one optical conduit, such as an optical fiber 506, is configured to enable endoscopic viewing of anatomy distal relative to the distal tip 566 of the balloon 560 during an example of use in accordance with the description herein.

The endoscope handle 502 may include any suitable components as would be apparent to one skilled in the art in view of the teachings herein. In some instances, the proximal endoscope handle 502 is configured to selectively couple with proximal end of the body 512 such that the handle assembly 510, a shaft assembly 550, and the balloon 560 act as a modular endoscope shaft operable to attach with the endoscope handle 502 prior to use.

The actuator 516 in the current example is slidably disposed over the body 512. Similar to the actuator 316, 416 described above, the actuator 516 is attached to a pull wire 570 that is substantially similar to the pull wire 370 described above. The pull wire 570 is configured to bend at least a distal portion of the shaft assembly 550 and the balloon 560 between a substantially straight configuration and a bent configuration.

The shaft assembly 550 includes a proximal outer shaft 552 attached to the handle assembly 510, an inner shaft 554 extending from the handle assembly 510 distally past the outer shaft 552, and a deflection guide 530 attached to a distal end of the outer shaft 552 such that the inner shaft 554 is contained within the deflection guide 530 but extends distally past the deflection guide 530. A luer fitting 520 extends from the body 512, which is substantially similar to the luer fitting 320, 420 described above. Therefore, the luer fitting 520 is configured to selectively couple with a suitable source of fluid. Additionally, the luer fitting 520 is in fluid communication with the inflation lumen (not shown) of the inner shaft 554 that terminates distally into communication with the interior of the balloon 560. Therefore, the luer fitting 520 and the inflation lumen (not shown) are configured to provide fluid communication between a suitable source of fluid and the interior of the balloon 560 in order to selectively inflate and deflate the balloon 560 in accordance with the description herein.

Distal the inner shaft 554 is attached to the balloon 560. The pull wire 570 is attached to a distal end of the balloon 560 and extends proximally from the balloon 560 along the length of the inner shaft 554. The pull wire 570 is configured to slide relative to the inner shaft 554. In some instances, distal the inner shaft 554 defines a pull wire lumen (not shown) that is substantially similar to the pull wire lumen 356 described above. The pull wire lumen (not shown) may slidably receive the pull wire 570, which extends from a distal portion of the balloon 560 all the way to the actuator 516 of the handle assembly 510. The pull wire 570 is fixed to the distal portion of the balloon 560 as well as the actuator 516. Therefore, a clinician may slide the actuator 516 proximally along the body 512 in order to acuate the pull wire 570 relative to the inner shaft 554, thereby deflecting the balloon 560 between the substantially straight configuration and the bent configuration. The balloon 560 of this example may thus transition between the substantially straight configuration and the bent configuration similar to the balloon 360 as described above with reference to FIGS. 10A-10B. The balloon 560 may be resiliently biased toward the straight configuration such that once tension from the pull wire 570 is relieved, the balloon 560 may bend itself back toward the straight configuration.

The inner shaft 554 may be sufficiently flexible such that a distal portion of the inner shaft 554 bends along with the balloon 560 between the straight configuration and the bent configuration. In some instances, it may be desirable to ensure the balloon 560 and the inner shaft 554 consistently bend along a predetermined path relative to the handle assembly 510 along a path that is transverse to the longitudinal axis of the dilation catheter 500.

As mentioned above, the shaft assembly 550 includes a deflection guide 530. The deflection guide 530 is fixed to the outer shaft 552 and extends distally from the outer shaft 552. The deflection guide 530 is configured to bend in response to the inner shaft 554 and the balloon 560 bending between the straight configuration and the bent configuration. Additionally, the deflection guide 530 is configured to bend along a predetermined path to thereby ensure the inner shaft 554 bends along a corresponding predetermined path in response to actuation of the pull wire 570 in accordance with the description herein.

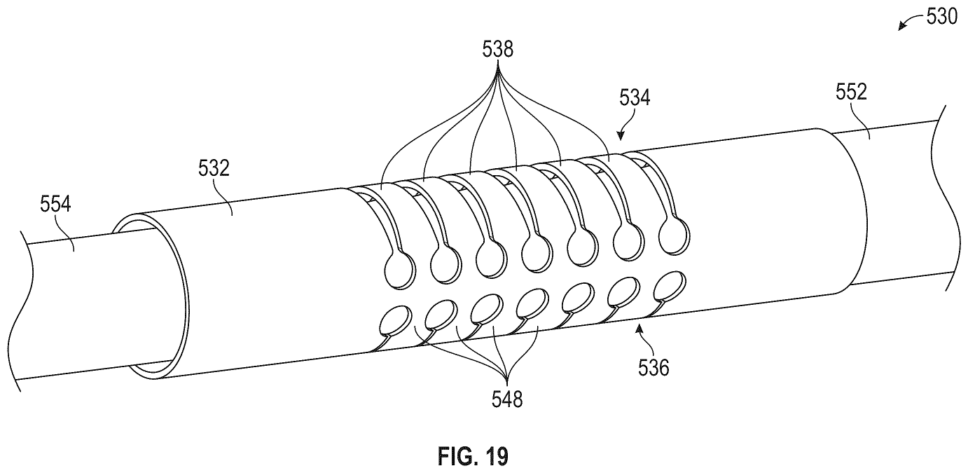

The deflection guide 530 includes a longitudinally extending tubular body 532 which houses a portion of the inner shaft 554. As best shown in FIG. 19, the longitudinally extending tubular body 532 includes a first array of ribs 538 defining a first plurality of circumferential cutouts 534 and a second array of ribs 540 defining a second plurality of circumferential cutouts 536. The first array of ribs 538 and the second array of ribs 540 are circumferentially opposed relative to each other on the tubular body 532. In other words, the first array of ribs 538 extends longitudinally and is located on a first lateral side of the tubular body 532; while the second array of ribs 540 extends longitudinally and is located on a second, opposite, lateral side of the tubular body 532.

The space between adjacent ribs 538 in the first array of ribs 538 (i.e., the circumferential cutouts 536) is larger compared to the space between adjacent ribs 540 in the second array of ribs 540 (i.e., the circumferential cutouts 536). This difference in spacing between adjacent ribs 538, 540 may lead to an increased flexibility to the half of the tubular body 532 associated with the first array of ribs 538 as compared to the flexibility of the half of the tubular body 532 associated with the second array of ribs 540. In other words, the ribs in the first array of ribs 538 are more prone to flexing toward each other, as compared to the ribs in the second array of ribs 540, in response to deflection of the inner shaft 554 in accordance with the description herein. Therefore, as the inner shaft 554 is deflected in response to actuation of the pull wire 570, the deflection guide 530 is configured to consistently bend along a predetermined path where the first array of ribs 538 flex toward each other, thereby guiding the inner shaft 554 to also bend along the same predetermined path.

It should be understood from the foregoing that the dilation catheter 500 is operable to be driven to bend laterally away from the central longitudinal axis of the proximal outer shaft 552 at two different longitudinal positions—at the longitudinal position of the balloon 560 and at the longitudinal position of the tubular body 532. In some versions, the dilation catheter 500 is operable to bend at both of these locations simultaneously. In some such versions, the same single pull-wire, set of pull-wires, or other actuator(s) is operable to drive the simultaneous bending at the balloon 560 and at the tubular body 532. In some other versions, the dilation catheter 500 is operable to bend independently at the balloon 560 and at the tubular body 532. In some such versions, one single pull-wire, set of pull-wires, or other actuator(s) is operable to drive bending at the balloon 560, while another single pull-wire, set of pull-wires, or other actuator(s) is operable to drive bending at the tubular body 532. Alternatively, in some versions where the dilation catheter 500 is operable to bend independently at the balloon 560 and at the tubular body 532, the same set of pull-wires may be operable to independently drive bending at the balloon 560 and at the tubular body 532. In still other versions, the dilation catheter 500 is only operable to be driven to bend at the tubular body 532, such that the dilation catheter 500 is not also operable to be driven to bend at the balloon 560.

While circumferential cutouts 534, 536 were utilized in the current example to increase the flexibility of one side of the tubular body 532 as compared to another, any other suitably means may be used as would be apparent to one skilled in the art in view of the teachings herein.

In the current example, the inner shaft 554 extends through the tubular body 532 and the outer shaft 552. However, in some instances, the tubular body 532 is interposed between the inner shaft 554 and the outer shaft 554 such that a proximal end of the flexible inner shaft 554 is attached to a distal end of the tubular body 532 and such that a distal end of the outer shaft 552 is attached is to a proximal end of the tubular body 532. In such instances, the pull wire 570 may slidably extend along/within the shafts 552, 554 and the tubular body 532 in order to couple to the actuating body 516.

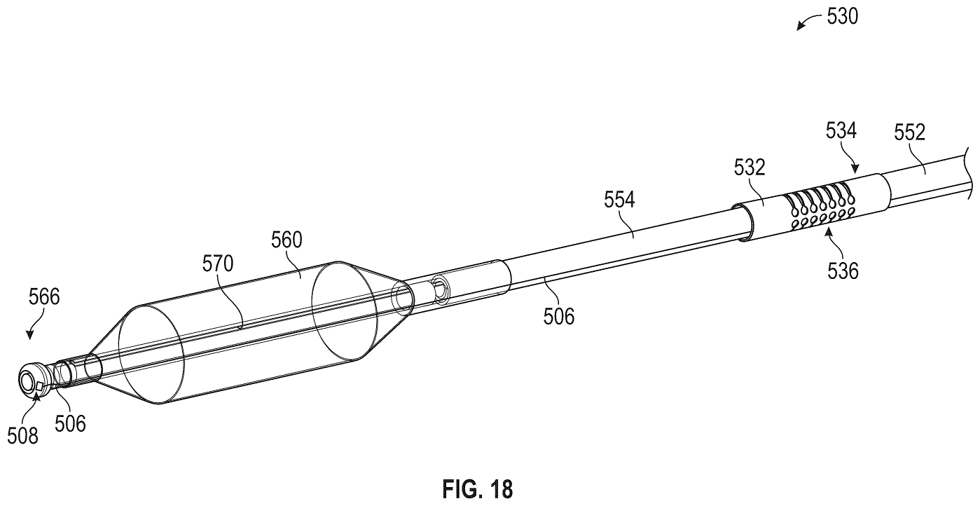

As mentioned above, the endoscope handle 502 is configured to selectively couple with a viewing monitor 504 such that images captured in accordance with the description herein may be viewed by a clinician at the viewing monitor 504. As best shown in FIG. 18, a distal tip 566 of the balloon 560 defines a viewing window 508. A distal end of the optical conduit 506 is located within or adjacent to the viewing window 508. The distal end of the optical conduit 506 is positioned relative to the viewing window 508 such that the optical conduit 506 may capture visual images of objects located distal relative to the distal tip 566 of the balloon 560. The optical conduit 506 extends proximally through the balloon 560, the shaft assembly 510, the handle assembly 510, and within the endoscope handle 502. A proximal end of the optical conduit 506 may be optically coupled with lens elements, an image sensor, and/or other optical components contained within the endoscope handle 502. Images captured via the viewing window 508, the optical conduit 506, and optical components contained within the endoscope handle 502 may be further communicated along a connector that is configured to couple with a processor that is operable to drive the display screen 504. The optical conduit 506, the endoscope handle 502, and/or the display screen 504 may have any suitable components to provide the functionality described above as would be apparent to one skilled in the art in view of the teachings herein.

In some versions, the optical conduit 506 includes an optical fiber. In some other versions, the optical conduit 506 includes one or more wires. In some such versions, a camera or other feature integrating an image sensor may be positioned at a distal end of the optical conduit 506. Such a camera may be similar to the camera 164 described above. In some such versions, the portion of the optical conduit 506 extending through the shaft assembly 550, the handle assembly 510, and the endoscope handle 502 may be substantially similar to the camera communication wire 163 described above. Additionally, the distal tip 566 of the balloon 560 may include light sources with communication wires extending proximally from the light sources, which may be substantially similar to the light source 166 and the communication wires 167 described above, respectively. Additionally, the distal tip 566 of the balloon 560 may be substantially similar to the annular frame 162 described above. Therefore, the visualization assembly of the deflectable dilation catheter 500 may include any suitable components as would be apparent to one skilled in the art in view of the teachings herein.

### III. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

Example 1. An apparatus, comprising: (a) a shaft dimensioned to be inserted transnasally to facilitate access to a Eustachian Tube of a pediatric patient, the shaft comprising: (i) a rigid proximal portion defining an open proximal end, (ii) a deflectable distal portion located distally from the proximal portion, the rigid proximal portion and the deflectable distal portion defining a lumen, and (iii) a pull wire extending from the deflectable distal portion along the proximal portion, the pull wire being configured to drive the deflectable distal portion between a straight configuration and a bent configuration; and (b) a visualizing distal tip located distally from the deflectable distal portion, the visualizing distal tip being configured to actuate with the deflectable distal portion between the straight configuration and the bent configuration, the visualizing distal tip comprising a distally facing camera, the visualizing distal tip defining a portion of the lumen; and the deflectable distal portion, while in the straight configuration, being configured to be inserted transnasally into a nasopharynx region of the pediatric patient, the deflectable distal portion being configured to be deflected from the straight configuration into the bent configuration while located within the nasopharynx region of the pediatric patient such that the visualizing distal tip is oriented toward the Eustachian Tube of the pediatric patient.

Example 2. The apparatus of Example 1, further comprising a handle assembly configured to be grasped by a single hand, the handle assembly comprising: (i) a first handle section, the first handle section fixed to the rigid proximal portion of the shaft, (ii) a second handle section slidably coupled to the first handle section, and (iii) a pull wire anchor fixed to both a proximal portion of the pull wire and the second handle section such that the first handle section and the second handle section are configured to actuate relative to each other to drive the deflectable distal portion between the straight configuration and the bent configuration.

Example 3. The apparatus of Example 2, the handle assembly further comprising a resilient member biasing the first handle section and the second handle section away from each other.

Example 4. The apparatus of Example 3, further comprising a camera communication wire extending proximally from the camera, along the shaft, and into an interior defined by the handle assembly.

Example 5. The apparatus of Example 4, the second handle section defining an elongate slot, the camera communication wire extending out of the interior defined by the handle assembly via the elongate slot.

Example 6. The apparatus of Example 5, the first handle section defining a chamber dimensioned to slidably housed a portion of the second handle section, a proximal section of the elongate slot being dimensioned to slide within the chamber as first handle section and the second handle section actuate toward each other.

Example 7. The apparatus of Example 6, the first handle section comprising a distally presented surface, the second handle section comprising a hard stop configured to directly engage the distally presented surface to thereby prevent a distal section of the elongate slot from sliding within the chamber.

Example 8. The apparatus of any of Examples 2 through 7, the first handle section comprising a collar fixed to the rigid proximal portion of the shaft.

Example 9. The apparatus of Example 8, the collar of the first handle section defining a pathway in communication with the open proximal end of the rigid proximal portion of the shaft.

Example 10. The apparatus of Example 9, the pathway of the first handle section and the open proximal end of the rigid proximal portion of the shaft being dimensioned to slidably receive a dilation catheter.

Example 11. The apparatus of any of Examples 1 through 10, the visualization distal tip further comprising a light source located adjacent to the camera.

Example 12. The apparatus of any of Examples 1 through 11, the visualizing distal tip further comprising a position sensor.

Example 13. The apparatus of any of Examples 1 through 12, further comprising a dilation catheter slidably disposed within the lumen.

Example 14. The apparatus of Example 13, the dilation catheter comprising a second pull wire configured to drive the dilation catheter between a straight configuration and a bent configuration.

Example 15. The apparatus of Example 13, the dilation catheter defining a working lumen, the working lumen housing a second camera.

Example 16. The apparatus of Example 15, the second camera being fixed within the working lumen of the dilation catheter.

Example 17. The apparatus of Example 15, the second camera being slidably disposed within the working lumen of the dilation catheter.

Example 18. The apparatus of any of Examples 15 through 17, further comprising a position sensor fixed relative to the second camera.

Example 19. An apparatus, comprising: (a) a shaft dimensioned to be inserted transnasally to facilitate access a Eustachian Tube of a pediatric patient, the shaft comprising: (i) a rigid proximal portion defining an open proximal end, (ii) a deflectable distal portion located distally from the proximal portion, the rigid proximal portion and the deflectable distal portion defining a lumen, and (iii) a first pull wire extending from the deflectable distal portion along the proximal portion, the first pull wire being configured to drive the deflectable distal portion between a straight configuration and a bent configuration; and (b) a dilation catheter slidably disposed within the lumen, the dilation catheter comprising: (i) a balloon, and (ii) a second pull wire configured to deflect the balloon between a straight configuration and a bent configuration, the deflectable distal portion and the balloon, while each being in the respective straight configurations, being configured to be inserted transnasally into a nasopharynx region of the pediatric patient, the deflectable distal portion and the balloon being configured to be deflected from the respective straight configurations into the respective bent configurations while located within the nasopharynx region of the pediatric patient such that deflectable distal portion is aligned with the Eustachian Tube of the pediatric patient.

Example 20. A method of accessing and dilating the Eustachian Tube of a pediatric patient, the method comprising: (a) inserting a deflectable distal portion of a guide catheter preloaded with a dilation catheter transnasally to access a nasopharynx region of the pediatric patient while the deflectable distal portion of the guide catheter is in a straight configuration; (b) actuating a handle assembly of the guide catheter in order to drive the deflectable distal portion from the straight configuration into a deflected configuration such that an open distal tip of the guide catheter is oriented toward the Eustachian Tube of the pediatric patient; (c) actuating a distal end of the dilation catheter distally past the open distal tip of the guide catheter until a dilator of the dilation catheter is within the Eustachian Tube of the pediatric patient; (d) dilating the Eustachian Tube of the pediatric patient via the dilator of the dilation catheter; (e) retracting the dilation catheter back into the guide catheter; (f) returning the deflectable distal portion from the deflected configuration back to the straight configuration; and (g) while the deflected distal portion is in the straight configuration, removing the guide catheter and the dilation catheter from the nasopharynx region of the pediatric patient.

Example 21. An apparatus, comprising: (a) a dilation catheter shaft having a proximal region and a distal region, the proximal region defining a longitudinal axis; (b) an inflatable balloon positioned along the distal region of the dilation catheter shaft, the inflatable balloon being configured to enter a Eustachian tube while in a deflated state, the inflatable balloon being configured to dilate the Eustachian tube while in the inflated state; (c) a deflectable portion positioned along the distal region of the dilation catheter shaft; and (d) an actuator operable to drive deflection of the distal region of the dilation catheter shaft away from the longitudinal axis, at the deflectable portion, while the proximal region of the dilation catheter shaft is positioned along the longitudinal axis.

Example 22. The apparatus of Example 21, the distal region having a distal tip, the distal tip having a bulbous configuration.

Example 23. The apparatus of any of Examples 21 through 22, the deflectable portion being coextensive with at least a portion of the inflatable balloon along the distal region.

Example 24. The apparatus of any of Examples 21 through 23, the actuator comprising a pull wire, the pull wire having a distal end secured to the distal region at a point distal to the deflectable portion.

Example 25. The apparatus of Example 24, further comprising an actuation assembly positioned at or proximal to the proximal region of the dilation catheter shaft.

Example 26. The apparatus of Example 25, the actuation assembly comprising a slider.

Example 27. The apparatus of Example 26, further comprising a handle secured to the proximal region of the dilation catheter shaft.

Example 28. The apparatus of Example 27, the handle comprising a body, the slider being operable to slide along the body.

Example 29. The apparatus of any of Examples 21 through 28, further comprising a guide catheter, the dilation catheter shaft being configured to slide within the guide catheter.

Example 30. The apparatus of Example 29, the guide catheter having a deflectable distal portion.

Example 31. The apparatus of Example 29, the guide catheter having a flexible distal portion.

Example 32. The apparatus of Example 29, the guide catheter having a rigid distal portion.

Example 33. The apparatus of Example 32, the rigid distal portion being straight.

Example 34. The apparatus of any of Examples 31 through 33, the dilation catheter shaft including a ventilation pathway extending along at least the distal region.

Example 35. The apparatus of any of Examples 31 through 34, further comprising a position sensor, the position sensor being configured to generate signals indicating a real-time position of the distal region in three-dimensional space.

Example 36. The apparatus of Example 21, further comprising a navigation sensor attached to a distal end of the inflatable balloon.

Example 37. The apparatus of Example 21, further comprising a deflection guide configured to guide the distal region of the dilation catheter along a predetermined path as the distal region of the dilation catheter shaft is deflected away from the longitudinal axis.

Example 38. The apparatus of Example 21, further comprising a camera element positioned at a distal end of the inflatable balloon.

Example 39. The apparatus of Example 38, further comprising a proximal endoscope handle.

Example 40. A method comprising: (a) inserting a deflectable distal portion of a dilation catheter transnasally to access a nasopharynx region of a pediatric patient while the deflectable distal portion of the dilation catheter is in a straight configuration; (b) actuating an actuator of the dilation catheter to drive the deflectable distal portion from the straight configuration into a deflected configuration such that a distal end of the dilation catheter is oriented toward a Eustachian Tube of the pediatric patient; (c) advancing the dilation catheter to position an inflatable balloon of the dilation catheter within the Eustachian Tube of the pediatric patient; (d) inflating the inflatable balloon of the dilation catheter to thereby dilate the Eustachian Tube of the pediatric patient; (e) deflating the inflatable balloon of the dilation catheter; (f) returning the deflectable distal portion from the deflected configuration back to the straight configuration; and (g) while the deflected distal portion is in the straight configuration, removing the dilation catheter from the nasopharynx region of the pediatric patient.

### IV. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, examples, examples, etc. described herein may be combined with any one or more of the other teachings, expressions, examples, examples, etc. that are described herein. The above-described teachings, expressions, examples, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various examples of the present inventions, further adaptations of the methods and systems described herein may be accomplished by appropriate modifications by one of ordinary skill in the art without departing from the scope of the present inventions. Several of such potential modifications have been mentioned, and others will be apparent to those skilled in the art. For instance, the examples, examples, geometrics, materials, dimensions, ratios, steps, and the like discussed above are illustrative and are not required. Accordingly, the scope of the present inventions should be considered in terms of the following claims and is understood not to be limited to the details of structure and operation shown and described in the specification and drawings.

What is claimed is:

1. A catheter system, comprising:
a shaft dimensioned to be inserted transnasally to facilitate access to a Eustachian Tube of a pediatric patient and comprising a rigid proximal portion defining an open proximal end, a deflectable distal portion located distally from the proximal portion, wherein the rigid proximal portion and the deflectable distal portion define a lumen, and a pull wire extending from the deflectable distal portion along the proximal portion and being configured to drive the deflectable distal portion between a straight configuration and a bent configuration; and
a visualizing distal tip located distally from the deflectable distal portion and configured to actuate with the deflectable distal portion between the straight configuration and the bent configuration, wherein the visualizing distal tip comprises a distally facing camera and defines a portion of the lumen,
wherein the deflectable distal portion is configured to be inserted transnasally into a nasopharynx region of the pediatric patient when the deflectable distal portion is in the straight configuration,
the deflectable distal portion is configured to be deflected from the straight configuration into the bent configuration when the deflectable distal portion is located within the nasopharynx region of the pediatric patient, such that the visualizing distal tip is oriented toward the Eustachian Tube of the pediatric patient.

2. The catheter system of claim 1, further comprising a handle assembly configured to be grasped by a single hand and comprising a first handle section fixed to the rigid proximal portion of the shaft, a second handle section slidably coupled to the first handle section, and a pull wire anchor fixed to both a proximal portion of the pull wire and the second handle section, such that the first handle section and the second handle section are configured to actuate relative to each other to drive the deflectable distal portion between the straight configuration and the bent configuration.

3. The catheter system of claim 2, wherein the handle assembly further comprises a resilient member configured to bias the first handle section and the second handle section away from each other.

4. The catheter system of claim 3, further comprising a camera communication wire extending proximally from the camera, along the shaft, and into an interior defined by the handle assembly.

5. The catheter system of claim 4, wherein the second handle section defines an elongate slot, and the camera communication wire extends out of the interior defined by the handle assembly via the elongate slot.

6. The catheter system of claim 5, wherein the first handle section defines a chamber dimensioned to slidably house a portion of the second handle section, and a proximal section of the elongate slot is dimensioned to slide within the chamber as the first handle section and the second handle section actuate toward each other.

7. The catheter system of claim 6, wherein the first handle section comprises a distally presented surface, and the second handle section comprises a hard stop configured to directly engage the distally presented surface to thereby prevent a distal section of the elongate slot from sliding within the chamber.

8. The catheter system of claim 7, wherein the first handle section comprises a collar fixed to the rigid proximal portion of the shaft.

9. The catheter system of claim 8, wherein the collar of the first handle section defines a pathway in communication with the open proximal end of the rigid proximal portion of the shaft.

10. The catheter system of claim 9, wherein the pathway of the first handle section and the open proximal end of the rigid proximal portion of the shaft are dimensioned to slidably receive a dilation catheter.

11. The catheter system of claim 10, wherein the visualization distal tip further comprises a light source located adjacent to the camera.

12. The catheter system of claim 11, wherein the visualizing distal tip further comprises a position sensor.

13. The catheter system of claim 12, further comprising a dilation catheter slidably disposed within the lumen.

14. The catheter system of claim 13, wherein the dilation catheter comprises a second pull wire configured to drive the dilation catheter between a straight configuration and a bent configuration.

15. The catheter system of claim 13, wherein the dilation catheter defines a working lumen that houses a second camera.

16. The catheter system of claim 15, wherein the second camera is fixed within the working lumen of the dilation catheter.

17. The catheter system of claim 15, wherein the second camera is slidably disposed within the working lumen of the dilation catheter.

18. The catheter system of claim 17, further comprising a position sensor fixed relative to the second camera.

19. A catheter system, comprising:
a shaft dimensioned to be inserted transnasally to facilitate access a Eustachian Tube of a pediatric patient and comprising a rigid proximal portion that defines an open proximal end, a deflectable distal portion located distally from the proximal portion, wherein the rigid proximal portion and the deflectable distal portion define a lumen, and a first pull wire extending from the deflectable distal portion along the proximal portion and being configured to drive the deflectable distal portion between a straight configuration and a bent configuration; and
a dilation catheter slidably disposed within the lumen and comprising a balloon and a second pull wire configured to deflect the balloon between a straight configuration and a bent configuration,
wherein the deflectable distal portion and the balloon are configured to be inserted transnasally into a nasopharynx region of the pediatric patient when the deflectable distal portion and the balloon are in the respective straight configurations,
wherein the deflectable distal portion and the balloon are configured to be deflected from the respective straight configurations into the respective bent configurations when the deflectable distal portion and the balloon are located within the nasopharynx region of the pediatric patient, such that deflectable distal portion is aligned with the Eustachian Tube of the pediatric patient.

20. A method of accessing and dilating a Eustachian Tube of a pediatric patient, the method comprising:
inserting a deflectable distal portion of a guide catheter preloaded with a dilation catheter transnasally to access a nasopharynx region of the pediatric patient while the deflectable distal portion of the guide catheter is in a straight configuration;
actuating a handle assembly of the guide catheter in order to drive the deflectable distal portion from the straight configuration into a deflected configuration such that an open distal tip of the guide catheter is oriented toward the Eustachian Tube of the pediatric patient;
actuating a distal end of the dilation catheter distally past the open distal tip of the guide catheter until a dilator of the dilation catheter is within the Eustachian Tube of the pediatric patient;
dilating the Eustachian Tube of the pediatric patient via the dilator of the dilation catheter;
retracting the dilation catheter back into the guide catheter;
returning the deflectable distal portion from the deflected configuration back to the straight configuration; and
while the deflected distal portion is in the straight configuration, removing the guide catheter and the dilation catheter from the nasopharynx region of the pediatric patient.
